(19) United States
(12) Patent Application Publication
(10) Pub. No.: US 2026/0294311 A1
(43) Pub. Date: Oct. 1, 2026

(54) SYSTEM FOR CONTROLLING A VEHICLE IN RESPONSE TO A DETECTED MEDICAL EVENT

(71) Applicant: Medtronic, Inc., Minneapolis, MN (US)

(72) Inventors: Kyle R. Zablocki, Jamesburg, NJ (US); Grant A. Neitzell, Loretto, MN (US)

(21) Appl. No.: 19/479,163

(22) PCT Filed: Mar. 27, 2024
(86) PCT No.: PCT/IB2024/052935

§ 371 Date: Oct. 27, 2025

Related U.S. Application Data
(60) Provisional application No. 63/498,441, filed on Apr. 26, 2023.

(51) Int. Cl. A61B 5/33; A61B 5/00; A61B 5/346; A61N 1/39; B60W 60/00

(52) U.S. Cl. CPC A61B 5/33; A61B 5/0006; A61B 5/0031; A61B 5/346; A61B 5/686; B60W 60/0016; B60W 60/0025; B60W 60/0051; A61N 1/3904; B60W 2540/221; B60W 2556/45

(57) ABSTRACT

An implantable medical device (IMD) comprising: one or more sensors; sensing circuitry configured to sense, via the one or more sensors, a physiological signal of a patient; communications circuitry configured to wirelessly communicate with computing circuitry of a vehicle; and processing circuitry configured to: determine that the patient is operating the vehicle; adjust, based on a determination that the patient is operating the vehicle, a threshold condition for the sensed electrical signal; determine, whether the sensed electrical signal satisfies the adjusted threshold condition; and in response to a determination that the sensed electrical signal satisfies the adjusted threshold condition, cause the communications circuitry to transmit instructions to the computing circuitry to cause the vehicle to engage an autonomous-operating mode.

This application claims the benefit of U.S. Provisional Patent Application Ser. No. 63/498,441, filed Apr. 26, 2023, the entire content of which is incorporated herein by reference.

### TECHNICAL FIELD

The disclosure relates generally to medical systems and, more particularly, medical systems configured to monitor patient activity for changes in patient health.

### BACKGROUND

Some types of medical systems may monitor various patient data, e.g., cardiac electrogram (EGM) data, of a patient to detect changes in health. In some examples, the medical system may monitor the cardiac EGM data to detect one or more types of arrhythmia, such as bradycardia, tachycardia (e.g., atrial tachycardia), fibrillation (e.g., atrial fibrillation), or asystole (e.g., caused by sinus pause or AV block). In some examples, the medical system may include one or more of an implantable medical device or a wearable device to collect the data.

### SUMMARY

Patients may experience various medical conditions at various times of the day. Some medical conditions may have effects on the patient that may incapacitate the patient and render the patient unable to perform various activities such as operating a motor vehicle. Incapacitating effects may include, cardiac arrest, loss of consciousness, degradation in situational awareness and environmental perception, loss of control of one or more limbs, or other effects that would renter a patient physically or mentally unable to operate a motor vehicle at a same level as a normal vehicle operator under identical external conditions. The medical conditions may have a short onset period (e.g., over a number of seconds, over a number of minutes) and/or may be relatively undetectable by the patient prior to the patient experiencing an incapacitating effect of the medical condition. In such examples, the patient may not have sufficient time to determine that they are experiencing the medical condition and navigate the vehicle to a safe location. In some scenarios, the patient may be incapacitated by the effects of the medical condition while the patient is actively operating the vehicle (e.g., while the patient is driving the vehicle), which increases the risk of a motor vehicle accident and a risk of harm to the patient, nearby motorists, and/or nearby pedestrians.

Medical systems and techniques as described herein include techniques to detect that a patient will experience a medical condition when the patient is operating a vehicle based on sensed physiological signals. The medical system may sense the physiological signals via one or more implantable medical devices (IMD) and/or one or more external wearable medical devices. The medical system may then control or instruct a vehicle with an autonomous-operation functionality to autonomously navigate the vehicle in an event of incapacitation of the patient as a result of the medical condition. The medical system may determine, based on the sensed physiological signals, a type or a severity of the medical condition and may cause the vehicle to autonomously navigate to a specific location and/or perform a specific function based on the determined type and severity of the medical condition. The vehicle may autonomously navigate to a complete stop, to one or more locations capable of rendering medical aid to the patient, output a request for aid to one or more parties, and/or perform other functions to render medical aid to the patient.

Medical systems and techniques described herein may use a machine learning model to more accurately determine whether a patient is experiencing a medical condition based on physiological data collected by a medical device. In some examples, the machine learning model is trained with a set of training instances, where one or more of the training instances comprise data that indicate relationships between a physiological parameter and classifications related to a medical condition, e.g., related to a type and/or severity of a medical condition experienced by the patient. Medical systems and techniques described herein may use a machine learning mode to more accurately determine an optimal action by an autonomous navigating vehicle in response to a type and severity of a medical condition a patient is experiencing. In some examples, the machine learning model is trained with a set of training instances, wherein one or more of the training instances comprise data that indicate relationships between a type and severity of a medical condition and a type of medical aid required by the patient. Because the machine learning models are trained with potentially thousands or millions of training instances, the machine learning models may, for example, reduce the amount of error in determining whether the patient is experiencing a type and/or severity of a medical condition and/or the type of medical aid required by the patient based on the type and/or severity of the medical condition, when compared to conventional medical systems.

The medical systems and techniques described herein may provide several advantages over other medical device systems. In some examples, the medical system adjusts threshold detection criteria for medical conditions based on a determination that the patient is operating a vehicle. Adjustments to the threshold detection criteria may allow for the faster detection of medical conditions and may provide the patient with a longer reaction time to the medical condition. In some examples, controlling the vehicle to autonomously navigate to different locations based on the type and severity of the medical condition may reduce a time between detection of the medical condition and delivery of medical aid or of a specific type of medical aid for the type and/or severity of the medical condition.

In some examples, the disclosure describes an implantable medical device (IMD) comprising: one or more sensors; sensing circuitry configured to sense, via the one or more sensors, a physiological signal of a patient; communications circuitry configured to wirelessly communicate with computing circuitry of a vehicle; and processing circuitry configured to: determine that the patient is operating the vehicle; adjust, based on a determination that the patient is operating the vehicle, a threshold condition for the sensed electrical signal; determine, whether the sensed electrical signal satisfies the adjusted threshold condition; and in response to a determination that the sensed electrical signal satisfies the adjusted threshold condition, cause the communications circuitry to transmit instructions to the computing circuitry to cause the vehicle to engage an autonomous-operating mode.

In some examples, the disclosure describes a vehicle comprising: control circuitry configured to autonomously operate the vehicle; communications circuitry configured to wirelessly communicate with an implantable medical device (IMD); and computing circuitry configured to: determine, via one or more sensors in communication with the computing circuitry, that a patient is operating the vehicle; receive, from the IMD and via the communications circuitry, an indication that the patient is experiencing a medical condition based on a determination that a physiological signal sensed by the IMD satisfies a threshold condition; determine, based at least in part on the received indication, a severity of the medical condition; select, based on a determined severity of the medical condition, an autonomous-operating mode from a plurality of autonomous-operating modes; and cause the control circuitry to autonomously operate the vehicle based on the selected autonomous-operating mode.

In some examples, the disclosure describes a method for controlling operation of an implantable medical device (IMD) comprising: sensing, by sensing circuitry included in a housing of the IMD and via one or more sensors coupled to the sensing circuitry, a physiological signal of a patient, wherein the sensing circuitry and the one or more sensors are operably coupled to a processing circuitry included in the housing of the IMD; determining, by the processing circuitry, whether the patient is operating a vehicle; adjusting, by the processing circuitry and based on a determination that the patient is operating the vehicle, a threshold condition for the sensed electrical signal; determining, by the processing circuitry, whether the sensed electrical signal satisfies the adjusted threshold condition; and based on a determination that the sensed electrical signal satisfies the adjusted threshold condition, transmitting, by the processing circuitry and via communications circuitry operably coupled to the processing circuitry, instructions to cause computing circuitry of the vehicle to engage an autonomous-operating mode.

In some examples, the disclosure describes a method for controlling operation of computing circuitry of a vehicle comprising: determining, by the computing circuitry and via one or more sensors in communication with the computing circuitry, that a patient is currently operating the vehicle; receiving, by the computing circuitry and via communications circuitry of the vehicle, an indication that a patient is experiencing a medical condition based on a determination that a physiological signal sensed by an implantable medical device (IMD) coupled to the patient satisfies a threshold condition; determining, by the computing circuitry and based at least in part on the received indication, a severity of the medical condition; selecting, by the computing circuitry and based on a determined severity of the medical condition, an autonomous-operating mode from a plurality of autonomous-operating modes; and causing, by the computing circuitry, control circuitry of the vehicle to autonomously operate the vehicle based on the selected autonomous-operating mode.

In some examples, the disclosure describes a computer-readable medium that, when executed by processing circuitry of an implantable medical device (IMD), is configured to cause the processing circuitry to perform a method for controlling operation of an implantable medical device (IMD) comprising: sensing, by sensing circuitry included in a housing of the IMD and via one or more sensors coupled to the sensing circuitry, a physiological signal of a patient, wherein the sensing circuitry and the one or more sensors are operably coupled to a processing circuitry included in the housing of the IMD; determining, by the processing circuitry, whether the patient is operating a vehicle; adjusting, by the processing circuitry and based on a determination that the patient is operating the vehicle, a threshold condition for the sensed electrical signal; determining, by the processing circuitry, whether the sensed electrical signal satisfies the adjusted threshold condition; and based on a determination that the sensed electrical signal satisfies the adjusted threshold condition, transmitting, by the processing circuitry and via communications circuitry operably coupled to the processing circuitry, instructions to cause computing circuitry of the vehicle to engage an autonomous-operating mode.

In some examples, the disclosure describes a computer-readable medium that, when executed by processing circuitry of an implantable medical device (IMD), is configured to cause the processing circuitry to perform a method for controlling operation of computing circuitry of a vehicle comprising: determining, by the computing circuitry and via one or more sensors in communication with the computing circuitry, that a patient is currently operating the vehicle; receiving, by the computing circuitry and via communications circuitry of the vehicle, an indication that a patient is experiencing a medical condition based on a determination that a physiological signal sensed by an implantable medical device (IMD) coupled to the patient satisfies a threshold condition; determining, by the computing circuitry and based at least in part on the received indication, a severity of the medical condition; selecting, by the computing circuitry and based on a determined severity of the medical condition, an autonomous-operating mode from a plurality of autonomous-operating modes; and causing, by the computing circuitry, control circuitry of the vehicle to autonomously operate the vehicle based on the selected autonomous-operating mode.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates an example medical system in conjunction with a patient, in accordance with one or more examples of the present disclosure.

FIG. 2 is a block diagram of an example implantable medical device of FIG. 1.

FIG. 3 is a block diagram of an example vehicle of FIG. 1.

FIG. 4 is a perspective drawing illustrating an implantable medical device of FIG. 1.

FIG. 5 is a perspective drawing illustrating another implantable medical device of FIG. 1.

FIG. 6 is a flowchart illustrating an example process of determining that a patient is experiencing a medical condition.

FIG. 7 is a flowchart illustrating an example process of autonomously navigating a vehicle based on a determination that a patient is experiencing a medical condition.

FIG. 8 is a flowchart illustrating another example process of autonomously navigating a vehicle based on a determination that a patient is experiencing a medical condition.

FIG. 9 is a flowchart illustrating another example process of autonomously navigating a vehicle based on a determination that a patient is experiencing a medical condition.

FIG. 10 is a conceptual diagram illustrating an example machine learning model configured to determine whether the patient is experiencing a medical condition.

FIG. 11 is a conceptual diagram illustrating an example training process for an artificial intelligence model, in accordance with examples of the current disclosure.

Like reference characters denote like elements throughout the description and figures.

### DETAILED DESCRIPTION

Some medical conditions may have effects on a patient that may at least temporarily incapacitate the patient when the patient experiences an onset or an episode of the medical condition. Example medical conditions may include, but are not limited to, cardiac conditions such as an arrhythmia (e.g., tachyarrhythmia), onset of a stroke, hypoglycemia, or other medical conditions. An incapacitated patient may be mentally and/or physically incapable of performing specific tasks such as operating a vehicle. For example, the effects of the medical condition can render the patient unconscious, disorientated, distracted, unable to control portions of their body (e.g., one or more limbs), or otherwise unable to safely and effectively operate a vehicle as compared to another motorist.

For some medical conditions, the patient may have sufficient reaction time (e.g., based on an indication by a medical system, based on the patient's own perception of symptoms or other physiological signs indicative of an imminent onset of the medical condition) to navigate the vehicle to a specific location (e.g., a medical care provider, an emergency medical service (EMS) provider, a location to park the vehicle without impeding or endangering other motorists) and/or to request medical aid. For other medical conditions, such as arrhythmia, the onset of a stroke, hypoglycemia, or the like, the patient may be unable to sense an imminent onset of the medical condition and/or a rapid onset of the medical condition may provide the patient insufficient reaction time between determining that the patient is experiencing the medical condition and the incapacitation of the patient due to the medical condition.

If the patient is incapacitated while operating the vehicle, the patient may lose control of the vehicle, which may increase the risk of a motor vehicle accident occurring and/or increase the risk of damage or harm to other individuals around the patient or the vehicle of the patient, e.g., other motorists, nearby pedestrians, or the like.

The devices, systems, and techniques described in this disclosure include techniques for detecting whether the patient is experiencing or will imminently experience a medical condition when the patient is operating a vehicle. A medical system may include a medical device (e.g., an implantable medical device (IMD), a wearable medical device) that may determine, based on sensed physiological parameters from the patient, that the patient is experiencing or will experience a medical condition. The physiological parameters may include, but are not limited, an electrocardiogram (ECG) signal from a heart of the patient, blood pressure levels of the patient, oxygen saturation levels of the patient, electrical signals in nerves of the patient, brain activity of the patient, or glucose levels of the patient. The medical device may compare values for one or more physiological parameters to threshold values corresponding to a medical condition and determine that the patient is experiencing or will experience the medical condition based on a determination that the sensed physiological parameter value(s) satisfy the corresponding threshold value(s).

Based on a determination that the patient is experiencing or will experience the medical condition, the medical system may cause the vehicle to autonomously navigate to a specific location. When the vehicle is autonomously navigating, the vehicle may self-operate without any input from the patient. For example, an autonomously navigating car is configured to drive along roads to and stop at specific locations without user input (e.g., on a steering wheel, on an accelerator, on a brake pedal, or the like). The vehicle may include control and computing systems configured to autonomously navigate the vehicle based on one or more autonomous navigation modes. The vehicle may select, based on the determined medical condition (e.g., based on a determined type and/or a determined severity of the medical condition), an autonomous navigation mode and autonomous operate based on the selected autonomous navigation mode. The vehicle may locate a specific location and autonomously navigate to the specific location. The specific location may be a parking spot that the vehicle may stop at without impeding or endangering other motorists, may be a nearby healthcare provider (e.g., a hospital, an emergency medical services (EMS) provider), or may be a nearby location of medical devices capable of rendering medical aid to the patient (e.g., a location with an automated external defibrillator (AED)). The vehicle may further autonomously request medical aid for the patient (e.g., via visual or auditory signals from the vehicle, from user(s) in a network).

The devices, systems, and techniques may provide one or more benefits over other medical monitoring systems. The medical system described herein may adjust threshold detection criteria for medical conditions (e.g., threshold values) based on a determination that the patient is operating a vehicle. Adjustments to the threshold detection criteria may allow for increased detection sensitivity and faster detection of medical conditions, thereby increasing detection accuracy and decreasing a likelihood of missing a medical condition. In some examples, controlling the vehicle to autonomously navigate to different locations based on different types and severities of the medical conditions may reduce a time between detection of the medical condition and delivery of medical aid or of a specific type of medical aid for the type and/or severity of the medical condition.

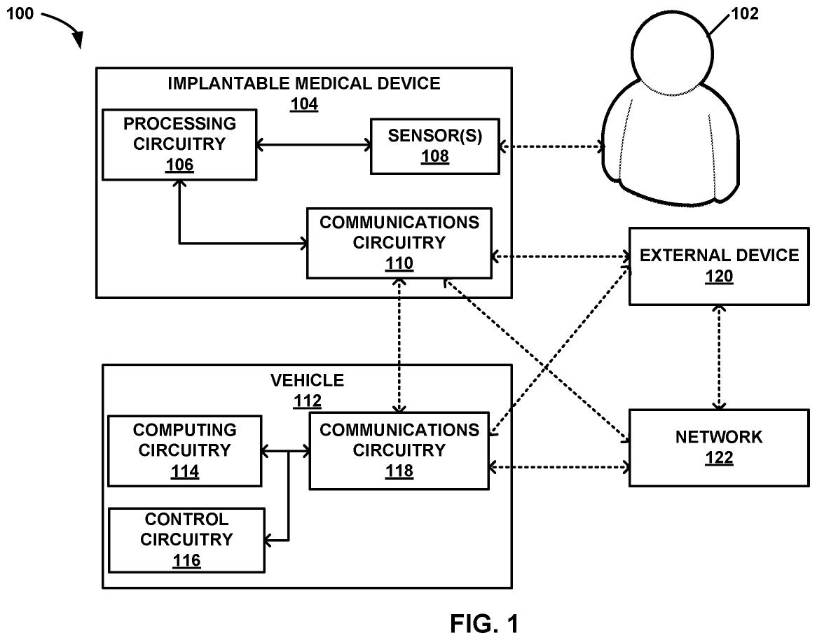

FIG. 1 illustrates an example medical system 100 in conjunction with a patient 102, in accordance with one or more examples of the present disclosure. System 100 includes an implantable medical device (IMD) 104 coupled to patient 102 and a vehicle 112 in communication with IMD 104 and an external device 120 and/or network 122. IMD 104 may include one or more computing modules including processing circuitry 106, sensor(s) 108, and communications circuitry 110. Vehicle 112 may include one or more computing modules including computing circuitry 114, control circuitry 116, and communications circuitry 118. While system 100 is described primarily with reference to a medical device of system 100 being IMD 104, another example system may include an external medical device (e.g., a wearable medical device) instead of or in addition to IMD 104. The external medical device may perform the same or similar function to IMD 104, as described in greater detail below.

IMD 104 may include one or more modules including, but are not limited to, processing circuitry 106, sensor(s) 108, and communications circuitry 110. IMD 104 may include, but are not limited to, an Implantable Cardiac Monitor (ICM), an Implantable Cardioverter Defibrillator (ICD), an implantable pacemaker, an implantable insulin pump, or an implantable glucose monitor. IMD 104 may be a Reveal LINQ™ or LINQ II™ ICM.

Processing circuitry 106 may sense, via sensor(s) 108, parameter values for one or more physiological parameters from patient 102. Sensor(s) 108 may include, but are not limited to, glucose monitors, electrodes (e.g., electrodes coupled to the heart of patient 102), accelerometers, oximeters, microphones, or optical sensors. Physiological parameters may include, but are not limited to, an electrocardiogram (ECG) signal from a heart of the patient 102, blood pressure levels of the patient 102, oxygen saturation levels of the patient 102, electrical signals in nerves of the patient 102, brain activity of the patient 102, and/or glucose levels of the patient 102. Physiological parameters may correspond to different medical conditions. For example, changes in parameter values may be correlative to an onset of a medical condition or a cessation of the medical condition. Each physiological parameter may correspond to one or more medical conditions. For example, ECG signals correspond to occurrence and cessation of arrhythmias. In some examples, glucose levels of patient 102 correspond to onset and cessation of hypoglycemia.

Processing circuitry 106 may continuously or periodically sense the parameter values from patient 102 and may determine changes in the parameter values and/or trends in the changes in the parameter values over time. Processing circuitry 106 may compare sensed parameter values, the determined changes in the parameter values (e.g., a magnitude of the change, a percentage change, a rate of change), and/or trends in the changes in the parameter values against threshold values of threshold conditions for physiological parameters. Each threshold condition includes one or more threshold values for one or more physiological parameters. For each physiological parameter, a threshold value may be a parameter value corresponding to an occurrence of a medical condition. Each physiological parameter may include multiple threshold values, each threshold value corresponding to a different type of medical condition and/or a different severity of a medical condition. For example, system 100 stores, for a threshold condition corresponding to an occurrence of arrhythmia, threshold detection criterion for arrhythmia. The threshold detection criterion may include, but is not limited to, a threshold heart rate, a threshold heartbeat duration, a heartbeat amplitude threshold of an ECG signal (e.g., for detection of R-waves), and/or a threshold number of intervals to detect (NID) value. A threshold NID value may correspond to a determination of an occurrence of an arrhythmia based on a consecutive number of heartbeats or a threshold percentage of heartbeats faster than a threshold heart rate, a first threshold ECG signal amplitude or a first threshold ECG signal frequency for a first severity level (e.g., for arrhythmia at a first severity score) and a second threshold ECG signal amplitude or a second threshold ECG signal frequency for a second severity level (e.g., for arrhythmia at a second severity score). Each severity level may be classified via a different severity score (e.g., via a different severity of illness (SOI) score).

Processing circuitry 106 may determine that patient 102 is experiencing or will experience a medical condition based on a determination that the sensed parameter values satisfy threshold values corresponding to the medical condition. Processing circuitry 106 may determine that patient 102 is experiencing the medical condition based on a determination that the sensed parameter values satisfy the threshold values for a threshold period of time. Processing circuitry 106 may determine a severity of the medical condition based on a determination that the sensed parameter values satisfy a threshold value corresponding to a specific severity level (e.g., corresponding to a specific SOI score).

The process of operating vehicle 112 may increase a likelihood of occurrence of a medical condition and/or increase a severity of the medical condition. Accordingly, processing circuitry 106 may adjust threshold conditions based on a determination that patient 102 is operating vehicle 112. For example, processing circuitry 106 may adjust (e.g., increase, decrease) threshold values for a threshold condition or adjust the threshold period of time required to satisfy the threshold condition. By adjusting threshold condition while patient 102 is operating vehicle 112, system 100 may increase the sensitivity of processing circuitry 106 to determine the occurrence of the medical condition. The increased sensitivity may decrease a number of actual occurrences of medical conditions that are not detected or are not detectable by the processing circuitry 106. Adjusting the threshold period of time may reduce an amount of time for IMD 104 to detect an occurrence of the medical condition. The reduced time may reduce time between onset of the medical condition and the autonomous navigation of vehicle 112, thereby further reducing a likelihood of an occurrence of an accident.

Processing circuitry 106 may determine that patient 102 is operating vehicle 112 based on a determination that communications circuitry 110 of IMD 104 has established wireless communications with communications circuitry 118 of vehicle 112. Processing circuitry 106 may receive, via communications circuitry 110, sensor data from one or more sensors of vehicle 112 (e.g., sensors inside a cabin of vehicle 112) confirming that patient 102 is operating vehicle 112 (e.g., that patient 102 is in the driver seat inside the cabin of vehicle 112, that patient 102 is interacting with a steering wheel of vehicle 112).

Processing circuitry 106 may adjust the threshold condition by a predetermined amount based on a determination that patient 102 is operating vehicle 112. The predetermined amount may be based on prior physiological parameter values from patient 102 and/or one or more other patients during operation of vehicle 112. In some examples, processing circuitry 106 may apply a machine learning model to determine the adjusted threshold values for the threshold condition. The machine learning model may be trained via a training set including physiological parameter values of patient 102 and/or one or more other patients while the patient(s) experience a specific type and/or severity of medical condition as the patient(s) operate a vehicle.

Processing circuitry 106 may transmit the determination that patient 102 is experiencing or will experience the medical condition to vehicle 112 (e.g., to computing circuitry 114 of vehicle 112). Processing circuitry 106 may also transmit a type and severity of the medical condition to vehicle 112. Computing circuitry 114 may, based on the received information from IMD 104, select an autonomous navigation mode from a plurality of autonomous navigation modes stored in vehicle 112. Each autonomous navigation mode may include instructions that, when executed by computing circuitry 114 cause control circuitry 116 of vehicle 112 to perform specific functions (e.g., to navigate vehicle 112 to specific locations and/or specific types of locations). For example, under a first autonomous navigation mode, control circuitry 116 controls components of vehicle 112 (e.g., engine, transmission, and steering of vehicle 112) to navigate to a location (e.g., a side of a road, a parking lot, an empty residential street) and park vehicle 112 at the location, e.g., to prevent collisions between vehicle 112 and other motorists or pedestrians.

Computing circuitry 114 may determine, based on the received information that the type and severity of the medical condition corresponds to scenario where patient 102 requires immediate medical aid. Computing circuitry 114 may then retrieve and execute a different autonomous navigation mode (e.g., a second autonomous navigation mode) In some examples, under the second autonomous navigation mode, computing circuitry 114 determines a route to a nearby a medical care facility (e.g., a EMS provider, healthcare provide, or medical care equipment (e.g., AES) location) and autonomously navigates vehicle 112 to the medical care location. Computing circuitry 114 may retrieve, from network 122 (e.g., a network storing global positioning system (GPS) information), a current location of vehicle 112 and a location of the medical care location. Computing circuitry 114 may then determine a route from the current location of vehicle 112 to the location of the medical care facility based on retrieved information from network 122). Control circuitry 116 may autonomously navigate vehicle 112 along the determined route to the medical care location.

In some examples, such as when computing circuitry 114 determines patient 102 is experiencing a medical condition that requires a specific type of medical aid (e.g., patient 102 is experiencing a stroke), computing circuitry 114 may retrieve and execute an autonomous navigation mode configured to cause control circuitry 116 to autonomously navigate vehicle 112 to a specialist medical care facility (e.g., a specialist medical center). The specific medical care facility may be further away than another medical care facility and vehicle 112 may bypass the closer medical care facility and navigate to the specialist medical care location.

Vehicle 112 may, in addition to or instead of autonomous navigating to a specific location, output one or more alerts to patient 102, another user, and/or a medical care provider. Vehicle 112 may output, via communications circuitry 118, an notification (e.g., a visual notification, an auditory notification) to patient 102 indicating that patient 102 is experiencing or will experience the medical condition, e.g., to provide patient 102 an opportunity to stop vehicle 112 at an isolated location. In some examples, vehicle 112 outputs, e.g., via alarm systems, warning lights, or vehicle horns, an alert to passing motorists and pedestrians requiring medical aid. In some examples, vehicle 112 transmits, to an external device 120 of a medical care provider, EMS provider, or family member of patient 102, a request for medical aid. In some examples, vehicle 112 transmit the request for medical aid to users of a network 122. The request for medical aid may include, but are not limited to, a location of patient 102, the severity and/or type of the medical condition, and/or other medical information for patient 102 (e.g., age, sex, allergies, prior medical history).

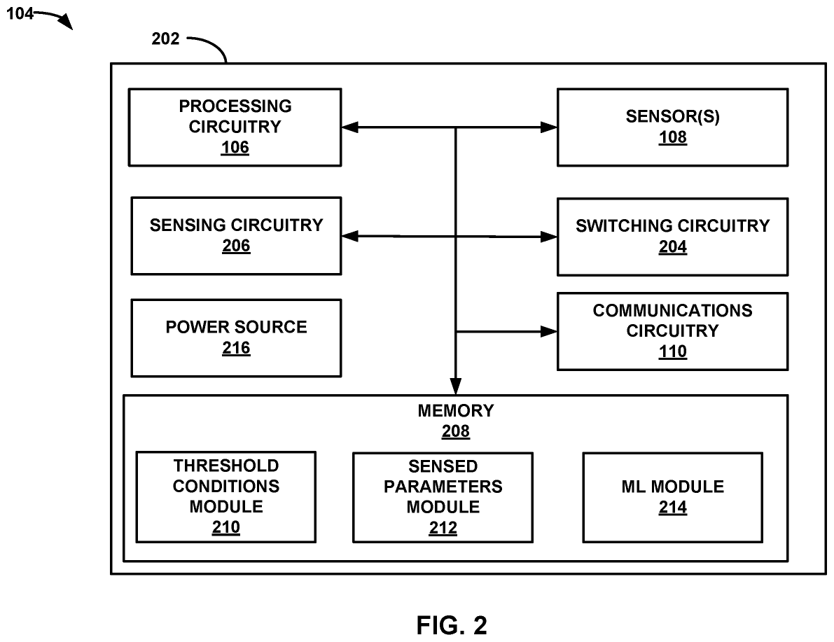

FIG. 2 is a block diagram of an example IMD 104 of FIG. 1. In the example illustrated in FIG. 2, IMD 104 includes processing circuitry 106, sensor(s) 108, communications circuitry 110, switching circuitry 204, sensing circuitry 206, memory 208 and power source 216 disposed within a housing 202 of IMD 104. The various circuitry may be or include programmable or fixed function circuitry configured to perform the functions attributed to respective circuitry. Memory 208 may store computer-readable instructions that, when executed by processing circuitry 106, cause IMD 104 to perform various functions. Memory 208 may be a storage device or other non-transitory medium. Memory 208 may include various modules configured to store different information. The various modules may include, but are not limited to, threshold conditions module 210 and sensed parameters module 212. Memory 208 may also store a machine learning (ML) model 214. Other examples of IMD 104 may include additional or different circuitry from the various circuitry described with respect to FIG. 2.

Switching circuitry 204 is coupled to sensor(s) 108. Sensor(s) 108 may include electrode(s), accelerometers, oximeters, glucose sensors, strain gauges, microphones, optical sensors, or other sensors configured to sense physiological signals from patient 102. Switching circuitry 204 may include one or more switch arrays, one or more multiplexers, one or more switches (e.g., a switch matrix of other collections of switches), one or more transistors, or other electrical circuitry. Switch circuitry 204 may be configured to direct electrical signals or other physiological signals from sensor(s) 108 to sensing circuitry 206, e.g., to sense physiological signals and parameter values for various physiological parameters from patient 102 (e.g., from blood of patient 102, from the heart of patient 102) via selected combinations of sensor(s) 108. The physiological parameters may include, but are not limited to, oxygen saturation, blood glucose levels, ECG signals, respiration rate, respiration patterns, or movement of patient 102.

Sensing circuitry 206 may include filters, amplifiers (e.g., sense amplifiers), analog-to-digital converters, capacitors, or other circuitry configured to sense signals (e.g., sensed electrical signals) from sensor(s) 108 and to convert the sensed signals to ECG signals, physiological signals, and/or physiological parameters. Sensing circuitry 206 may transmit the ECG signals, physiological signals, and/or physiological parameters to processing circuitry 106 and/or to memory 208, e.g., for storage in sensed parameters module 212 of memory 208.

Processing circuitry 106 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 106 herein may be embodied as firmware, hardware, software, or any combination thereof.

Processing circuitry 106 may determine whether patient 102 is currently experiencing or will experience a medical condition (e.g., a stroke, a hypoglycemic episode, arrhythmia). Processing circuitry 106 may receive sensed physiological signals and/or physiological parameter values from sensing circuitry 206. Processing circuitry 106 may retrieve threshold conditions from memory 208 (e.g., from threshold conditions module 210 of memory 208) and may compare the sensed physiological signals and/or physiological parameter values against threshold values for the same physiological parameters for the threshold conditions to determine whether the sensed signals from patient 102 satisfy any of the retrieved threshold conditions.

Each threshold condition may correspond to a different type of medical condition, For example, a first threshold condition corresponds to stroke, a second threshold condition corresponds to tachyarrhythmia, and a third threshold condition corresponds to hypoglycemia. In some examples, different threshold conditions correspond to different severity levels for a same medical condition. For example, the second threshold condition corresponds to tachyarrhythmia at a first severity and a fourth threshold condition corresponds to tachyarrhythmia at a second severity different than the first severity.

Each threshold condition may include corresponding threshold values (e.g., minimum threshold values, maximum threshold values) for one or more physiological parameters. For example, the second threshold condition corresponding to tachyarrhythmia includes threshold amplitudes and threshold frequencies for an ECG signal of the heart of patient 102. The threshold values for the physiological parameters correlate to an occurrence of the medical condition corresponding to the threshold condition at the severity level corresponding to the threshold condition. Processing circuitry 106 may determine that a threshold condition is satisfied by determining that the received physiological signals and/or physiological parameter values satisfy one or more or all of the threshold values for the threshold condition for a threshold period of time. For example, processing circuitry 106 determines that a threshold condition for an arrhythmia is satisfied by determining that IMD 104 did not detect cardiac activity from patient 102 for a threshold period of time (e.g., at least 5 seconds). Processing circuitry 106 may determine that a threshold condition is satisfied by determining that the received physiological signals and/or physiological parameter values satisfy one or more or all of the threshold values for the threshold condition, for a threshold number of instances. For example, processing circuitry 106 determines that a threshold condition for an arrhythmia is satisfied by determining that the heart of patient 102 is beating at rate outside a threshold range of heart rates for a threshold number of consecutive abnormal heartbeats (e.g., at least 16 abnormal heartbeats).

Based on a determination that patient 102 is experiencing or will experience a medical condition, processing circuitry 106 may transmit the determination to vehicle 112 via communications circuitry 110. Processing circuitry 106 may transmit a type and severity of the medical condition. In some examples, processing circuitry 106 transmits the physiological signals and/or physiological parameter values to vehicle 112, external device 120 and/or network 122. Processing circuitry 106 may transmit instructions to vehicle 112 to cause vehicle 112 to automatically navigate to a specific location (e.g., a parking location, a medical care location).

Processing circuitry 106 may adjust one or more threshold conditions based on a determination that patient 102 is operating the vehicle 112. During operation of vehicle 112, patient 102 may be more prone to experience a medical condition and the threshold conditions corresponding to medical conditions may change accordingly. Processing circuitry 106 may adjust threshold period of time to satisfy the threshold condition, e.g., to reduce an amount of time for IMD 104 to determine an occurrence of a medical condition. Processing circuitry 106 may determine that patient 102 is operating the vehicle 112 by determining that communications circuitry 110 is within a communications range of communications circuitry 118 and/or has established a wireless communication link or wireless communications channel with communications circuitry 118. Communications circuitry 110 may establish a direct communications link with communications circuitry 118 of vehicle 112 or may establish an indirect communications link communications circuitry 118 via an intermediate device. The intermediate device may include, but are not limited to, a smartphone, a smartwatch, a tablet, a laptop, an external programmer for IMD 104, or another computing device and/or system connected to patient 102. Communications circuitry 110 may communicate with communications circuitry 118 via the intermediate device without establishing a communications session with communications circuitry 118, and vice versa.

Processing circuitry 106 may determine that patient 102 is operating the vehicle 112 by receiving, via communications circuitry 110, a communication from communications circuitry 118 of vehicle 112 indicating that the vehicle 112 is power on. In some examples, processing circuitry 106 may receive, via communications circuitry 110, communications from another computing device that vehicle 112 is powered on and/or that patient 102 is currently operating the vehicle 112. Processing circuitry 106 may distinguish patient 102 operating vehicle 112 from patient 102 merely being within vehicle 112 by requesting a confirmation from patient 102 (e.g., via an external computing device, via a user interface of vehicle 112), or from vehicle 112 via one or more sensors within vehicle 112. For example, vehicle 112 confirms, in response to a request from IMD 104, that patient 102 is sitting in the driver's seat of vehicle 112 via one or more sensors disposed within the cabin of the vehicle. Vehicle 112 then transmits the confirmation to IMD 104 to confirm that patient 102 is operating vehicle 112.

Based on a determination that patient 102 is operating vehicle 112, processing circuitry 106 may adjust threshold values, threshold periods of time, and/or threshold number of instances for one or more threshold conditions. Adjustments to the threshold conditions (e.g., adjustments to threshold values, threshold periods of time, and/or threshold number of instances for the threshold condition) may increase the sensitivity of IMD 104 to detect a medical condition. Processing circuitry 106 may adjust the threshold conditions to predetermined values stored in memory 208. The predetermined values may be stored in threshold conditions module 210 and may correspond to previously determined medical condition detection threshold values while patient 102 is operating vehicle 112. The predetermined values may be based on previously recorded physiological signals and/or physiological parameter values from patient 102 and/or one or more other patients. In some examples, processing circuitry 106 retrieves and executes a machine learning model from memory 208 (e.g., from ML model 214), wherein the machine learning model is trained to output adjust values for threshold conditions in response to input data. The input data may include, but are not limited to, the physical condition of patient 102, prior recorded physiological signals and/or physiological parameters, the type of vehicle 112 (e.g., aircraft, watercraft, automobile, motorcycle), type and severity of the medical condition associated with the threshold condition, and/or current threshold condition values. The machine learning model may be trained using a training set including prior values for the input data and the corresponding recorded threshold condition values for an actual occurrence of the medical condition.

In some examples, processing circuitry 106 may adjust the threshold conditions while patient 102 is operating vehicle 112. For example, processing circuitry 106 adjusts the threshold conditions in response to a determination that patient 102 is operating vehicle 112 at increased speeds, at decreased speeds, under particular weather conditions, or the like. When vehicle 112 travels at faster speeds and/or under different environmental conditions, system 100 may have reduced time from the occurrence of the medical condition to detect the medical condition and autonomously navigate vehicle 112 in response to the detection, e.g., as compared to vehicle 112 traveling at slower speeds or under normal environmental conditions. Processing circuitry 106 may adjust the threshold conditions to increase sensitivity of IMD 104 to detect medical conditions and/or reduce the time for IMD 104 to detect medical conditions, e.g., to reduce the risk of collision while vehicle 112 is operating at different speeds and/or under different environmental conditions.

In some examples, e.g., due to the adjusted threshold conditions, processing circuitry 106 may determine occurrences of medical conditions which are false positives. Processing circuitry 106 may verify the validity of the determination of the occurrence of the medical condition. Processing circuitry 106 may continue to receive current physiological signals and/or physiological parameter values from sensing circuitry 206 and determine whether the received signals and/or parameter values correspond to the occurrence of the medical condition. Processing circuitry 106 may request and receive sensor data from vehicle 112. The sensor data may include data from sensors disposed within the cabin of vehicle 112 and include audio, visual, and/or tactile information on patient 102 within vehicle 112. Processing circuitry 106 may determine whether the sensor data indicate that patient 102 is experiencing symptoms of the medical condition. For example, processing circuitry 106 determines, based on the sensor data from vehicle 112, whether patient 102 is moving, swaying, shaking, in pain, or other showing any outward symptoms or effects of the medical condition. In some examples, processing circuitry 106 applies machine learning model 214 stored in memory 208 to determine whether patient 102 is exhibiting outward symptoms of the medical condition based on an input of the sensor data from vehicle 112. The machine learning model may be trained via a training set including audio, visual, or tactile sensor data of patient 102 and/or one or more other patients (e.g., data from numerous patients/subjects) and a corresponding indication of a type and/or severity of symptom(s) the patient(s) are experiencing when the sensor data is recorded.

Processing circuitry 106 may store physiological signals and/or physiological parameters values corresponding to an instance of a false-positive determination of an occurrence of the medical condition in memory 208. Processing circuitry 106 may compare the results of future determinations against false-positive determinations stored in memory 208 to determine whether the future determinations are false positives. Processing circuitry 106 may upload false-positive determinations and the corresponding signal and/or parameter values to network 122. The false-positive determinations and corresponding data may be used by other computing devices, computing systems, or cloud computing environments, e.g., to monitor the physiological health of patient 102 and/or to provide treatment to patient 102.

Vehicles 112 with autonomous navigation functionality may require user interaction with the vehicle 112 at a specific rate. For example, a vehicle 112 requires a user to touch a steering wheel of vehicle 112 every threshold number of seconds (e.g., every four seconds) and outputs a notification based on a determination that the user did not touch the steering wheel for at least the threshold number of seconds. Processing circuitry 106 may request and receive data from vehicle 112 corresponding to the interaction between patient 102 and vehicle 112. Processing circuitry 106 may confirm a determination of an occurrence of a medical condition based on a level of interaction between patient 102 and vehicle 112 (e.g., an amount of time between instances of patient 102 touching the steering wheel).

In some examples, the physiology and/or the disease state of patient 102 may change over time. Processing circuitry 106 may receive, e.g., from network 122, information corresponding to changes in the physiology or the disease state of patient 102 any may adjust the threshold conditions, e.g., to specific values determined by a clinician.

Communications circuitry 110 (alternatively referred to as “telemetry circuitry 110”) supports wireless communication between IMD 104 and vehicle 112, external device 120, and/or network 122. Processing circuitry 106 may provide information (e.g., physiological signals, physiological parameter values) to vehicle 112, external device 120, and/or network 122 via communications circuitry 110. Processing circuitry 106 may receive or retrieve information from vehicle 112, external device 120, and/or network 122 (e.g., sensor data from vehicle 112, patient information from external device 120 or network 122) via communications circuitry 110. Communications circuitry 110 may accomplish communication by wireless communication techniques such as radiofrequency (RF) communication techniques, e.g., via an antenna (not shown). Communications circuitry 110 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

In some examples, the components of IMD 104 may include computing components of a Reveal LINQ™ or LINQ II™ cardiac monitor available from Medtronic, Inc., Minneapolis, Minnesota. In such examples, IMD 104 may be capable of communicating directly or indirectly (e.g., via vehicle 112, via external device 120) with network 122 such as the CareLink™ Network available from Medtronic, Inc., Minneapolis, Minnesota, e.g., to detect medical conditions, store sensed signals and/or parameter values, and transmit sensed signals, sensed parameter values, and/or other information on detected medical conditions across multiple computing devices.

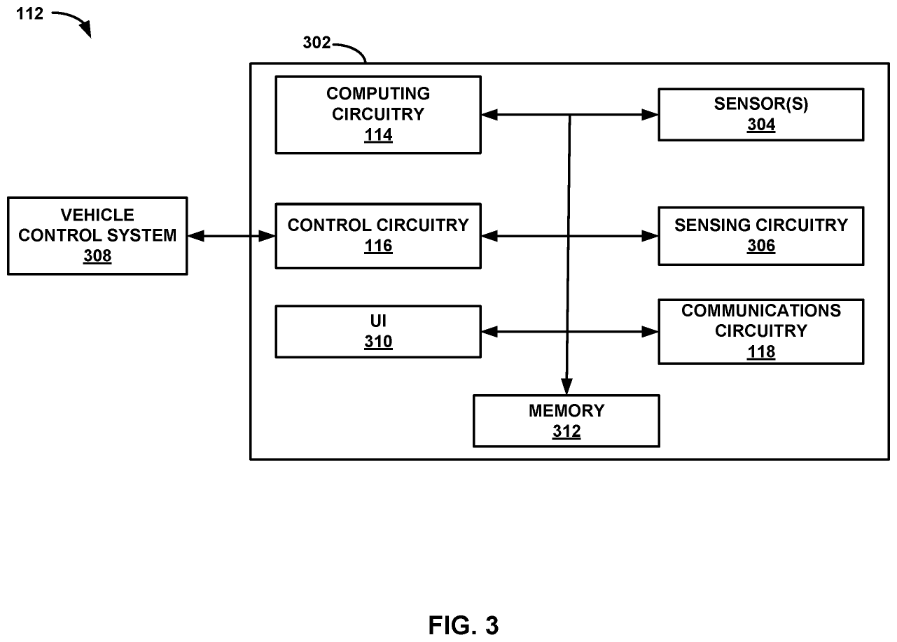

FIG. 3 is a block diagram of an example vehicle 112 of FIG. 1. Vehicle 112 may include a vehicle computing system 302 configured to control a vehicle control system 308 of vehicle 112 to operate vehicle 112. Vehicle computing system 302 may include computing circuitry 114, control circuitry 116, communications circuitry 118, sensor(s) 304, sensing circuitry 306, user interface (UI) 310, and memory 312. Memory 312 may store computer-readable instructions that, when executed by processing circuitry 106, cause IMD 104 to perform various functions. Memory 312 may be a storage device or other non-transitory medium. Memory 312 may include various modules configured to store different information. The vehicle computing system 302 may include other circuitry instead of or in addition to the various circuitry and components described herein.

A vehicle control system 308 of vehicle 112 may include one or more systems and/or components configured to move vehicle 112 (e.g., to propel, turn, rotate, and/or stop vehicle 112). Vehicle control system 308 may include or may be coupled to a propulsion mechanism of vehicle 112 (e.g., an engine of vehicle 112, a transmission of vehicle 112, an ignition system of vehicle 112), steering system(s) of vehicle 112, braking system(s) of vehicle 112, illumination systems of vehicle 112 (e.g., lights of vehicle 112), or to horns and/or alarm systems of vehicle 112. The vehicle control system 308 may be coupled to vehicle computing system 302, e.g., to control circuitry 116 of vehicle computing system 302. Vehicle control system 308 may receive and operate one or more systems and/or components and/or in response to instructions or commands from vehicle computing system 302 to move and/or operate vehicle 112 in accordance with the instructions or commands from vehicle computing system 302.

Vehicle computing system 302 may autonomously navigate vehicle 112. Vehicle computing system 302 may apply an autonomous navigation mode and transmit instructions or commands to vehicle control system 308 to move vehicle 112 in accordance with the applied autonomous navigation mode.

Computing circuitry 114 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to computing circuitry 114 herein may be embodied as firmware, hardware, software, or any combination thereof.

Computing circuitry 114 may be configured to select and apply an autonomous navigation mode. Computing circuitry 114 may select and apply autonomous navigation mode in response to receiving, via communications circuitry 118, a notification from IMD 104, external device 120, and/or network 122 that patient 102 is experiencing or will experience a medical condition.

The notification from IMD 104 may include a type and/or a severity of the medical condition. For example, a notification from IMD 104 may indicate that patient 102 is experiencing an arrhythmia at a specific SOI score. Computing circuitry 114 may select an autonomous navigation mode of a plurality of navigation modes stored in memory 312 based on the type and severity of the medical condition. Each combination of the type and severity of medical conditions may correspond to an autonomous navigation mode. Each store autonomous navigation mode may correspond to one or more different combinations of the type and severity of medical conditions. For example, occurrences of arrhythmias at different SOI scores correspond to different stored autonomous navigation modes. Each autonomous navigation mode may include metadata or other identification indicating the types and severities of medical conditions corresponding to the autonomous navigation mode. Computing circuitry 114 may select the autonomous navigation mode by matching the received medical condition information to the identification of one or more of the stores autonomous navigation modes.

In some examples, computing circuitry 114 may generate a severity score for the medical condition of patient 102 based on the type and severity of the medical condition. For example, computing circuitry 114 assigns a higher severity score for arrhythmia compared to hypoglycemia at a same severity level or compared to arrhythmia at a lower severity level. Each autonomous navigation mode may correspond to a range of severity scores and computing circuitry 114 may select an autonomous navigation mode by comparing the determined severity score against the corresponding severity scores for the stored autonomous navigation modes.

Each autonomous navigation mode may include instructions to cause control circuitry 116 to control vehicle control system 308 to autonomously perform one or more functions with one or more systems of vehicle 112 independent of any action or inaction by the drive (e.g., by patient 102). The one or more functions may include, but are not limited, outputting an alert to the drive, outputting an alert to nearby motorists and bystanders, navigating vehicle 112 to an isolated location (e.g., a location out of the path of any nearby motorists), stopping vehicle 112 at the isolated location, navigating vehicle 112 to a medical care location, or the like. A medical care facility may include, but are not limited, a location with a medical care provider, a location with a medical device (e.g., an AED), a hospital, an EMS service provider, a specialist medical center specialized in treating a particular medical condition or the like. Some autonomous navigation modes may be configured to only perform a single functions. Some autonomous navigation modes may be configured to perform two or more functions. In such example, computing circuitry 114 may select one function of the two or more functions and control vehicle control system 308 to perform the selected function in accordance with the instructions stored in the autonomous navigation mode.

Control circuitry 116 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to computing circuitry 114 herein may be embodied as firmware, hardware, software, or any combination thereof.

Control circuitry 116 may be coupled to one or more systems of vehicle control system 308. In response to receiving instructions from computing circuitry 114 and/or from patient 102 (e.g., via UI 310 or another control interface such as the steering column of vehicle 112), control circuitry 116 may manipulate the one or more system of vehicle control system 308 to cause vehicle 112 to move in a manner in accordance with the instructions from computing circuitry 114. In some examples, control circuitry 116 receives an intended movement of vehicle 112 from computing circuitry 114 and determines the specific inputs to one or more systems of vehicle control system 308 based on the intended movement. For example, in response to receiving an intention to move vehicle 112 from a first point to a second point, control circuitry 116 determine specific changes to the acceleration and steering of vehicle 112 to cause vehicle 112 to move from the first point to the second point. While FIG. 3 illustrates computing circuitry 114 and control circuitry 116 as different circuitry within vehicle computing system 302, the functions of both computing circuitry 114 and control circuitry 116 may be performed by a single computing component.

In accordance with some autonomous navigation modes, computing circuitry 114 may cause UI 310 to output a notification alerting patient 102. In such examples, the medical condition may have a low severity level or there may sufficient reaction time (e.g., minutes, hours) to allow patient 102 to navigate vehicle 112 to an isolated location and/or to a medical care location. Computing circuitry 114 may cause UI 310 and/or another computing device associated with patient 102 to output a notification to patient 102. The notification may indicate a type and severity of the determined medical condition. The notification may provide recommended courses of action to patient 102 (e.g., proceed to an isolated location, proceed to a medical care location) and may provide recommended routes to accomplish each course of action. Computing circuitry 114 may retrieve, e.g., via communications circuitry 118, location data (e.g., GPS data) corresponding to a current location of vehicle 112 and the final destinations for the recommended courses of action. Computing circuitry 114 may then determine, the recommended route to each final destination based on the retrieve location data. In some examples, computing circuitry 114 requests and receives the recommended routes from an external network or cloud computing environment (e.g., from network 122).

In accordance with some autonomous navigation modes, computing circuitry 114 may cause, via control circuitry 116, vehicle control system 308 to navigate vehicle 112 to and stop at an isolated location. The isolated location may be any location out of the line of travel of any motorists, pedestrians, or other bystanders. Isolated locations may include, but are not limited to, parking lots, driveways, sides of roads, grass fields, the sides of empty streets, or the like. Navigating vehicle 112 to the isolated location may reduce a risk of a collision between vehicle 112 and bystanders and may provide time for patient 102 to recover or to notify medical care providers. Computing circuitry 114 may identify isolated location via received location data and/or via sensor data transmitted by sensing circuitry 306 to computing circuitry. Computing circuitry 114 may identify, based on data from sensor(s) 304 coupled to sensing circuitry 306 (e.g., external cameras on vehicle 112), that a nearby location (e.g., a portion of a road or street ahead of vehicle 112) is an isolated location. Computing circuitry 114 may transmit instructions to control circuitry 116 to cause control circuitry 116 to control one or more systems of vehicle control system 308 (e.g., a steering system, a throttle system, a braking system) to autonomously navigate vehicle 112 to the isolated location.

In accordance with some autonomous navigation modes, computing circuitry 114 may transmit instructions to control circuitry 116 to cause one or more systems of vehicle control system 308 to transmit a request for medical aid. The request for medical aid may include, but are not limited to, flashing lights, audio signals (e.g., from a vehicle horn of vehicle 112), or the like. In some examples, computing circuitry 114 may transmit a request for medical aid to external device 120 and/or to network 122 via communications circuitry 118 of vehicle 112. Computing circuitry 114 may transmit, via network 122, the request to one or more users connected to network 122. The one or more users may include medical care providers.

In accordance with some autonomous navigation modes, computing circuitry 114 may transmit instructions to control circuitry 116 to control one or more systems of vehicle control system 308 to navigate to a medical care location. In some situations, the type and severity of the medical condition may necessitate the provision of immediate medical assistance to patient 102. In such examples, computing circuitry 114 may control vehicle 112 to autonomously navigate vehicle 112 to a medical care location, e.g., to reduce the time to render medical assistance to patient 102. Medical care facilities may include, but are not limited, locations with medical care providers (e.g., medical clinics, hospitals), EMS providers, or locations with medical devices configured to deliver an appropriate medical aid to patient 102. For example, computing circuitry 114 may select, based on a received determination from IMD 104 that patient 102 is experiencing an arrhythmia, that any nearby location with an AES as a medical care location. Computing circuitry 114 cause control circuitry 116 to navigate vehicle 112 to a specific medical care facility of a plurality of medical care facilities based on the type of medical care provided at the location, the distance of the location to vehicle 112, or based on prior inputs (e.g., by a clinician, by patient 102) indicating a preference for or against the location.

Computing circuitry 114 may select, based on the type and severity of the medical condition, a specific medical care facility from a plurality of nearby medical care facility and cause control circuitry 116 to navigate vehicle 112 to the specific medical care facility along a determined route. For example, computing circuitry 114 may, based on a determination that patient 102 is experiencing a stroke, cause control circuitry 116 to navigate vehicle 112 to a medical center specializing in treatment strokes. Computing circuitry 114 may navigate vehicle 112 to the specific medical care facility even though there are other medical care facilities that are closer to the location of vehicle 112, e.g., to provide more appropriate medical aid to patient 102.

In some examples, computing circuitry 114 may transmit, via communications circuitry 118, a notification to a computer device, system, or cloud computing environment of a specific medical care facility vehicle 112 is currently navigating towards. The notification, may include, but are not limited to, medical and/or biographical information of patient 102, the type and severity of the medical condition, emergency contacts of patient 102, an estimated time of arrival, a make and model of vehicle 112, and/or other information to facilitate the delivery of medical aid to patient 102 when vehicle 112 arrives at the specific medical care location.

Computing circuitry 114 may transmit, via communications circuitry 118, a notification to a computing device of a clinician, family members, and/or caretakers of patient 102. The notification may indicate a type and severity of the medical condition and action(s) taken by system 100 in response to the determination.

Sensing circuitry 306 may be coupled to sensor(s) 304 on vehicle 112. Sensing circuitry 306 may include filters, amplifiers (e.g., sense amplifiers), analog-to-digital converters, capacitors, or other circuitry configured to sense signals from an environment surrounding vehicle 112 and/or inside a cabin of vehicle 112 via sensor(s) 304. Sensor(s) 304 may be disposed within the cabin of vehicle 112 and/or on the outside of vehicle 112 (e.g., on a front bumper or a rear bumper of vehicle 112, around the sides of vehicle 112). Sensor(s) 304 may include, but are not limited to, cameras, microphones, pressure sensors, or proximity sensors.

In some examples, sensing circuitry 306 determines location, movement, or posture of patient 102 within vehicle 112 and transmits the sensed information to IMD 104 and/or to computing circuitry 114. Computing circuitry 114 may determine, based on the sensed information, whether patient 102 is experiencing any symptoms. Computing circuitry 114 may verify a received determination of an occurrence of a medical condition based on a determination that patient 102 is experiencing symptoms consistent with the type and severity of the medical condition.

In some examples, sensing circuitry 306 determines traffic, obstacles, and/or other environmental information via sensor(s) 304 disposed on the outside of vehicle 112. Sensing circuitry 306 may transmit the sensed information to computing circuitry 114. Computing circuitry 114 may autonomously navigate vehicle 112 based at least in part on the sensed information from sensing circuitry 306.

UI 310 may be configured to transmit information to patient 102 and receive inputs from patient 102. UI 310 may include one or more video and/or audio displays disposed on the steering column, on the dashboard, and/or on a center console of vehicle 112. UI 310 may receive user inputs from patient 102 and transmit the inputs to computing circuitry 114 and/or control circuitry 116. UI 310 may receive tactile, visual, or auditory inputs via a touchscreen, a camera, a microphone, switches, dials, or buttons.

Communications circuitry 118 (alternatively referred to as “telemetry circuitry 118”) supports wireless communication between vehicle 112 and IMD 104, external device 120, and/or network 122. Communications circuitry 118 may accomplish communication by wireless communication techniques such as radiofrequency (RF) communication techniques, e.g., via an antenna (not shown). Communications circuitry 118 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

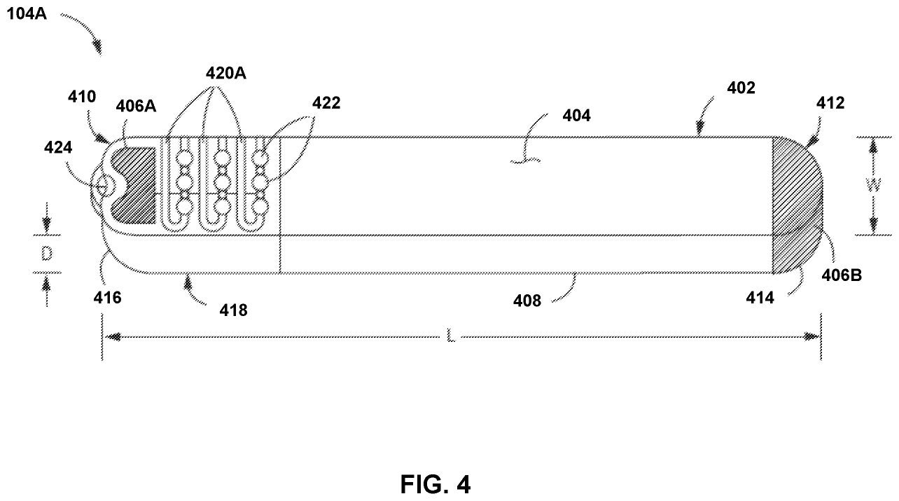

FIG. 4 is a perspective drawing illustrating an IMD 104A, which may be an example configuration of IMD 104 of FIG. 1 as an ICM. In the example shown in FIG. 4, IMD 104A may be embodied as a monitoring device having housing 402, proximal electrode 406A and distal electrode 406B. Housing 402 may further comprise first major surface 404, second major surface 408, proximal end 410, and distal end 412. Housing 402 encloses electronic circuitry located inside the IMD 104A and protects the circuitry contained therein from body fluids. Housing 402 may be hermetically sealed and configured for subcutaneous implantation. Electrical feedthroughs provide electrical connection of electrodes 406A and 406B.

In the example shown in FIG. 4, IMD 104A is defined by a length L, a width W and thickness or depth D and is in the form of an elongated rectangular prism wherein the length L is much larger than the width W, which in turn is larger than the depth D. In one example, the geometry of the IMD 104A—in particular a width W greater than the depth D—is selected to allow IMD 104A to be inserted under the skin of the patient 102 using a minimally invasive procedure and to remain in the desired orientation during insertion. For example, the spacing between proximal electrode 406A and distal electrode 406B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 104A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 404 may range from 3 mm to 15, mm, from 3 mm to 10 mm, or from 5 mm to 15 mm, and may be any single or range of widths between 3 mm and 15 mm. The thickness of depth D of IMD 104A may range from 2 mm to 15 mm, from 2 mm to 9 mm, from 2 mm to 5 mm, from 5 mm to 15 mm, and may be any single or range of depths between 2 mm and 15 mm. In addition, IMD 104A according to an example of the present disclosure is has a geometry and size designed for ease of implant and patient comfort. Examples of IMD 104A described in this disclosure may have a volume of three cubic centimeters (cm) or less, 1.5 cubic cm or less or any volume between three and 1.5 cubic centimeters.

In the example shown in FIG. 4, once inserted within the patient 102, the first major surface 404 faces outward, toward the skin of the patient 102 while the second major surface 408 is located opposite the first major surface 404. In addition, in the example shown in FIG. 4, proximal end 410 and distal end 412 are rounded to reduce discomfort and irritation to surrounding tissue once inserted under the skin of the patient 102. IMD 104A, including instrument and method for inserting IMD 104 is described, for example, in U.S. Pat. No. 11,311,312 filed on Mar. 11, 2014, granted on Apr. 26, 2022, and entitled “Subcutaneous Delivery Tool,” the entirety of which is incorporated herein by reference in its entirety.

Proximal electrode 406A is at or proximate to proximal end 410, and distal electrode 406B is at or proximate to distal end 412. Proximal electrode 406A and distal electrode 406B are used to sense cardiac EGM signals, e.g., ECG signals, thoracically outside the ribcage, which may be sub-muscularly or subcutaneously. EGM signals may be stored in a memory of IMD 104A, and data may be transmitted via integrated antenna 420A to vehicle 112, external device 120, or network 122. In some example, electrodes 406A and 406B may additionally or alternatively be used for sensing any bio-potential signal of interest, which may be, for example, an EGM, EEG, EMG, or a nerve signal, or for measuring impedance, from any implanted location.

In the example shown in FIG. 4, proximal electrode 406A is at or in close proximity to the proximal end 410 and distal electrode 406B is at or in close proximity to distal end 412. In this example, distal electrode 406B is not limited to a flattened, outward facing surface, but may extend from first major surface 404 around rounded edges 414 and/or end surface 416 and onto the second major surface 408 so that the electrode 406B has a three-dimensional curved configuration. In some examples, electrode 406B is an uninsulated portion of a metallic, e.g., titanium, part of housing 402.

In the example shown in FIG. 4, proximal electrode 406A is located on first major surface 404 and is substantially flat, and outward facing. However, in other examples proximal electrode 406A may utilize the three dimensional curved configuration of distal electrode 406B, providing a three dimensional proximal electrode (not shown in this example). Similarly, in other examples distal electrode 406B may utilize a substantially flat, outward facing electrode located on first major surface 404 similar to that shown with respect to proximal electrode 406A.

The various electrode configurations allow for configurations in which proximal electrode 406A and distal electrode 406B are located on both first major surface 404 and second major surface 408. In other configurations, such as that shown in FIG. 4, only one of proximal electrode 406A and distal electrode 406B is located on both major surfaces 404 and 408, and in still other configurations both proximal electrode 406A and distal electrode 406B are located on one of the first major surface 404 or the second major surface 408 (e.g., proximal electrode 406A located on first major surface 404 while distal electrode 406B is located on second major surface 408). In another example, IMD 104A may include electrodes on both major surface 404 and 408 at or near the proximal and distal ends of the device, such that a total of four electrodes are included on IMD 104A. Electrodes 406A and 406B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 4, proximal end 410 includes a header assembly 418 that includes one or more of proximal electrode 406A, integrated antenna 420A, anti-migration projections 422, and/or suture hole 424. Integrated antenna 420A is located on the same major surface (i.e., first major surface 404) as proximal electrode 406A and is also included as part of header assembly 418. Integrated antenna 420A allows IMD 104A to transmit and/or receive data. In other examples, integrated antenna 420A may be formed on the opposite major surface as proximal electrode 406A, or may be incorporated within the housing 402 of IMD 104A. In the example shown in FIG. 4, anti-migration projections 422 are located adjacent to integrated antenna 420A and protrude away from first major surface 404 to prevent longitudinal movement of the device. In the example shown in FIG. 4, anti-migration projections 422 include a plurality (e.g., nine) small bumps or protrusions extending away from first major surface 404. As discussed above, in other examples anti-migration projections 422 may be located on the opposite major surface as proximal electrode 406A and/or integrated antenna 420A. In addition, in the example shown in FIG. 4, header assembly 418 includes suture hole 424, which provides another means of securing IMD 104A to the patient 102 to prevent movement following insertion. In the example shown, suture hole 424 is located adjacent to proximal electrode 406A. In one example, header assembly 418 is a molded header assembly made from a polymeric or plastic material, which may be integrated or separable from the main portion of IMD 104A.

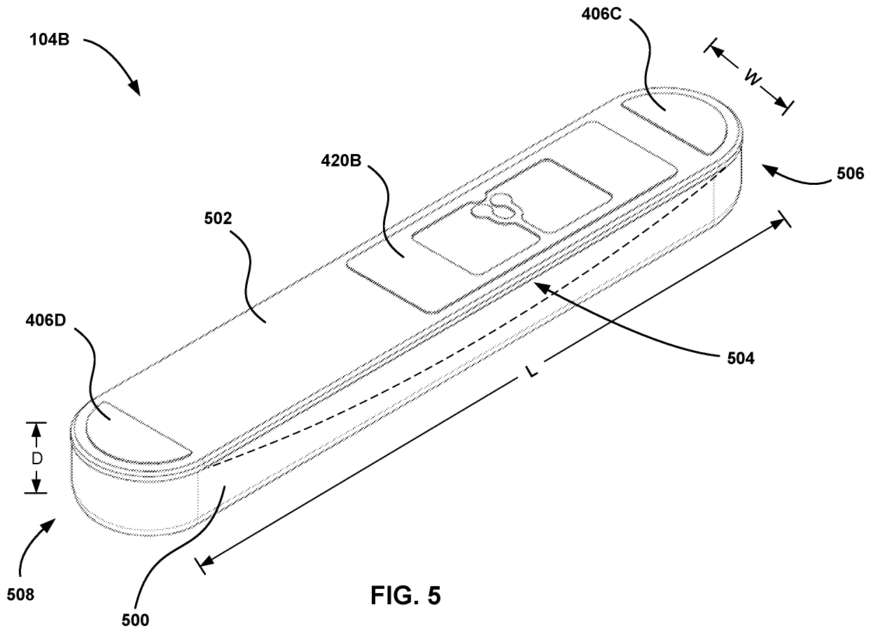

FIG. 5 is a perspective drawing illustrating another IMD 104B, which may be another example configuration of IMD 104 from FIG. 1 as an ICM. IMD 104B of FIG. 5 may be configured substantially similarly to IMD 104A of FIG. 4, with differences between them discussed herein.

IMD 104B may include a leadless, subcutaneously-implantable monitoring device, e.g. an ICM. IMD 104B includes housing having a base 500 and an insulative cover 502. Proximal electrode 406C and distal electrode 406D may be formed or placed on an outer surface of housing 402. Various circuitries and components of IMD 104B may be formed or placed on an inner surface of cover 502, or within base 500. In some examples, a battery or other power source of IMD 104B may be included within base 500. In the illustrated example, antenna 420B is formed or placed on the outer surface of base 500, but may be formed or placed on the inner surface in some examples. In some examples, insulative cover 502 may be positioned over an open base 500 such that base 500 and cover 502 enclose the circuitries and other components and protect them from fluids such as body fluids. The housing including base 500 and insulative cover 502 may be hermetically sealed and configured for subcutaneous implantation.

Circuitries and components may be formed on the inner side of insulative cover 502, such as by using flip-chip technology. Insulative cover 502 may be flipped onto a base 500. When flipped and placed onto base 500, the components of IMD 104B formed on the inner side of insulative cover 502 may be positioned in a gap 504 defined by base 500. Electrodes 406C and 406D and antenna 420B may be electrically connected to circuitry formed on the inner side of insulative cover 502 through one or more vias (not shown) formed through insulative cover 502. Insulative cover 502 may be formed of sapphire (i.e., corundum), glass, parylene, and/or any other suitable insulating material. Base 500 may be formed from titanium or any other suitable material (e.g., a biocompatible material). Electrodes 406C and 406D may be formed from any of stainless steel, titanium, platinum, iridium, or alloys thereof. In addition, electrodes 406C and 406D may be coated with a material such as titanium nitride or fractal titanium nitride, although other suitable materials and coatings for such electrodes may be used.

In the example shown in FIG. 5, the housing of IMD 104B defines a length L, a width W and thickness or depth D and is in the form of an elongated rectangular prism wherein the length L is much larger than the width W, which in turn is larger than the depth D, similar to IMD 104A of FIG. 4. For example, the spacing between proximal electrode 406C and distal electrode 406D may range from 5 mm to 50 mm, from 30 mm to 50 mm, from 35 mm to 45 mm, and may be any single spacing or range of spacings from 5 mm to 50 mm, such as approximately 40 mm. In addition, IMD 104B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 104B may range from 2 mm to 15 mm, from 5 mm to 15 mm, or from 3 mm to 5 mm, and may be any single depth or range of depths between 2 mm and 15 mm, such as approximately 4 mm. IMD 104B may have a volume of three cubic centimeters (cm) or less, or 1.5 cubic cm or less, such as approximately 1.4 cubic cm.

In the example shown in FIG. 5, once inserted subcutaneously within the patient, outer surface of housing 402 faces outward, toward the skin of the patient. In addition, as shown in FIG. 5, proximal end 506 and distal end 508 are rounded to reduce discomfort and irritation to surrounding tissue once inserted under the skin of the patient. In addition, edges of IMD 104B may be rounded.

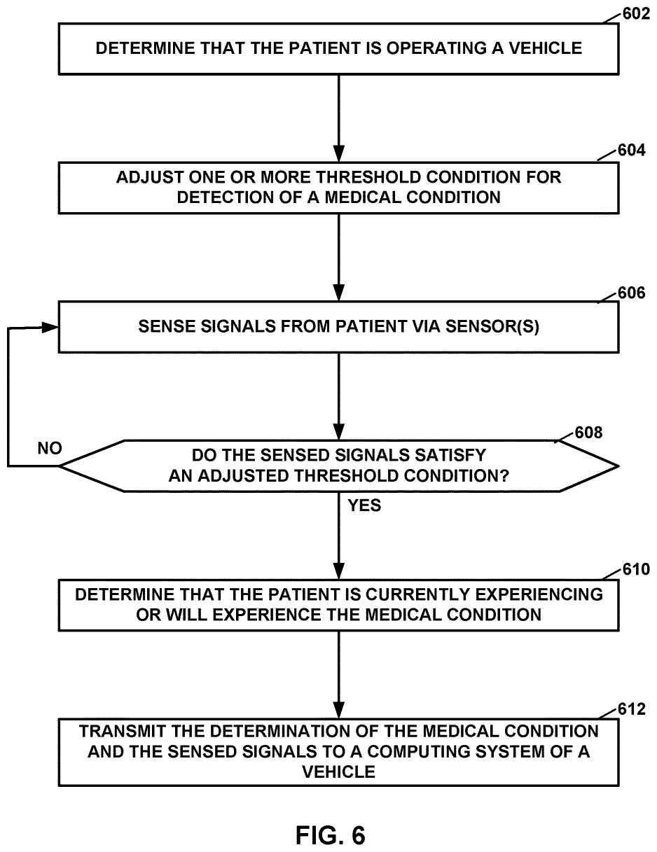

FIG. 6 is a flowchart illustrating an example process of determining that a patient 102 is experiencing a medical condition. While the example process illustrated in FIG. 6 are primarily described herein with respect to an implantable medical device (e.g., IMD 104), the example process may be performed by an external medical device and/or one or more other medical devices of medical system 100.

System 100 may determine that patient 102 is operating a vehicle 112 (602). IMD 104 may determine that patient 102 is operating vehicle 112 based on a determination that communications circuitry 110 of IMD 104 is within a communications range and/or is in wireless communication with vehicle 112 (e.g., with communications circuitry 118 of vehicle 112). IMD 104 may determine that patient 102 is operating vehicle 112 by determining that vehicle 112 is power on. IMD 104 may determined that vehicle 112 is powered on based on the establishment of a wireless communications link or a wireless communications channel between communications circuitry 110 of IMD 104 and communications circuitry 118 of vehicle 112. IMD 104 may establish a communications channel with vehicle 112 via an intermediate device (e.g., via a smartwatch, smartphone, tablet, laptop, external programmer for IMD 104, or other computing devices and/or computing systems connected to patient 102). A communications channel between IMD 104, the intermediate device, and vehicle 112 may allow IMD 104 to communicate with vehicle 112 without directly establishing a communications session with vehicle 112. IMD 104 may determine or verify that patient 102 is operating vehicle 112 by determining, via sensor(s) 304 in a cabin of vehicle 112, that patient 102 is in a driver's seat and/or is operating a steering column of vehicle 112.

IMD 104 may adjust one or more threshold conditions for the detection of a medical condition (604). Each threshold condition may correspond to an occurrence of a different type and/or severity of a medical condition. Medical conditions may include, but are not limited to, arrhythmias, strokes, hypoglycemic episodes, or other medical conditions that may mentally or physically incapacitate patient 102 and render patient 102 mentally or physically unable to operate vehicle 112. IMD 104 may determine an occurrence of a medical condition based on a determination that sensed physiological signals and/or physiological parameter values satisfy threshold values(s) of physiological parameters for a threshold condition (e.g., for a threshold period of time, for a threshold number of instances). Compared to determination during normal activities, IMD 104 may detect medical conditions at an increased sensitivity and/or at a faster rate, e.g., to reduce a risk of a vehicular accident resulting from loss of control of vehicle 112 by patient 102 due to effects of the medical condition.

IMD 104 may adjust a threshold condition by adjusting one or more of the threshold value(s), the threshold period of time, and/or the threshold number of instances for the threshold condition. IMD 104 may adjust the threshold condition based on predetermined values stored in IMD 104. IMD 104 may apply a machine learning model to input data for a threshold condition to output adjustments to one or more of threshold value(s), the threshold period of time, or the threshold number of instances. The input data may include, but are not limited to, the physical condition of patient 102, prior recorded physiological signals and/or physiological parameters, the type of vehicle 112 (e.g., aircraft, watercraft, automobile, motorcycle), type and severity of the medical condition associated with the threshold condition, and/or the current values for the threshold condition. The machine learning model may be trained via a training set including example values for one or more types of input data and the type and severity of the medical condition corresponding to the example values.

IMD 104 may sense signals from patient 102 via sensor(s) 108 (606). Sensing circuitry 206 may sense physiological signals from one or more sensor(s) 108 disposed on or within patient 102. IMD 104 may convert the sensed signals to physiological parameter values. Sensor(s) 108 may include, but are not limited to, electrodes, accelerometers, oximeters, strain gauges, microphones, optical sensors, or the like.

IMD 104 may determine whether the sensed physiological signals and/or the sensed physiological parameter values satisfy an adjusted threshold condition (608). IMD 104 may determine that the sensed signals satisfy an adjust threshold condition based on a determination that the sensed signals satisfy an adjusted threshold value of one or more physiological parameter for a threshold period of time and/or for a threshold number of instances. The adjusted threshold value may represent a threshold magnitude, a threshold frequency, a threshold change in the value of, a threshold rate of change in the value of, the presence of, and/or the absence of the physiological parameter. For example, IMD 104 determines that a threshold condition for arrhythmia is satisfied based on a determination that IMD 104 does not detect a threshold level of cardiac activity for a threshold period of time and/or for a threshold number of beats.

Based on a determination that the sensed signals do not satisfy an adjusted threshold condition (“NO” branch of 608), IMD 104 may continue to sensed signals from patient 102 via sensor(s) 108 (606). Based on a determination that the sensed signals satisfy the adjusted threshold condition (“YES” branch of 608), IMD 104 may determine that patient 102 is currently experiencing or will experience a medical condition corresponding to the adjusted threshold condition (610). IMD 104 may determine a severity level for the threshold condition. Each threshold condition may correspond to a different level of severity (e.g., a different SOI score) for a medical condition. IMD 104 may determine a severity of the medical condition based on the adjusted threshold condition(s) satisfied by the sensed signals. For example, IMD 104 determines that patient 102 is experiencing arrhythmia at a first severity level based on a determination that sensed cardiac activity of patient 102 satisfies an adjusted threshold condition for a first severity level but not an adjusted threshold condition for second severity level.

IMD 104 may retrieve sensor data from vehicle 112 to confirm that patient 102 is experiencing the medical condition. The sensor data from vehicle 112 may correspond to sensor data from sensor(s) 304 disposed within a cabin of vehicle 112. IMD 104 may determine whether the sensor data indicate that patient 102 is experiencing the symptoms indicative or corresponding to the determined medical condition at the determined severity. If IMD 104 does not verify that patient 102 is experiencing the determined medical condition, IMD 104 may store the determination and the corresponding sensed signals as a false-positive determination. IMD 104 may store false-positive determinations in memory 208 of IMD 104 and/or transmit the false-positive determinations to external device 120 and/or to network 122.

IMD 104 may transmit the determination of the medical condition and the sensed signals to a computing system (e.g., vehicle computing system 302) of vehicle 112 (612). Vehicle computing system 302 may autonomously navigate vehicle 112 in response to the determination of the medical condition, as will be described in greater detail below. IMD 194 may transmit the determination and the sensed signals to vehicle 112 via communications circuitry 110 of IMD 104. IMD 104 may transmit the determination and the sensed signals to external device 120 and/or to network 122.

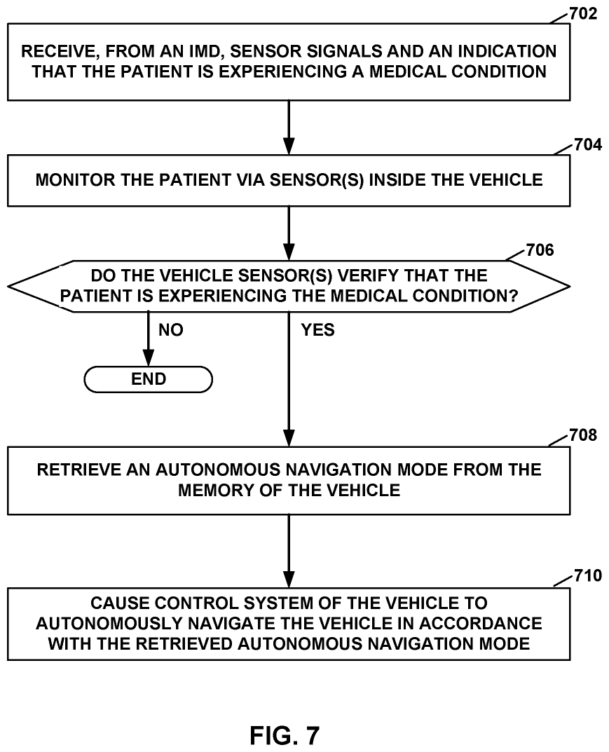

FIG. 7 is a flowchart illustrating an example process of autonomously navigating a vehicle 112 based on a determination that a patient 102 is experiencing a medical condition (702). A vehicle computing system 302 of vehicle 112 may receive, from an IMD (e.g., IMD 104), sensor signals and an indication that patient 102 is experiencing a medical condition. Vehicle computing system 302 may receive the indication and the sensor signals via communications circuitry 119 of vehicle computing system 302. The indication may indicate a type and a severity of the medical condition as determined by IMD 104.

Vehicle computing system 302 may monitor the patient 102 via sensor(s) 304 inside vehicle 112 (704). Sensor(s) 304 may be disposed within a cabin of vehicle 112 and may be configured to monitor behavior and posture of patient 102 while patient 102 is operating vehicle 112. Sensor(s) 304 may include, but are not limited to, cameras, microphones, pressure sensors, motion sensors, proximity sensors, or the like.

Vehicle computing system 302 may determine whether sensor(s) 304 verify that patient 102 is experiencing the medical condition (706). Vehicle computing system 302 may determine whether sensor data from sensor(s) 304 indicate that patient 102 is experiencing symptoms indicative of the type and severity of the medical condition as transmitted by IMD 104. Based on a determination that patient 102 is not experiencing the symptoms, vehicle computing system 302 may determine that patient 102 is not experiencing the medical condition (“NO” branch of 706), and may end the process (“END”).

Based on a determination that patient 102 is experiencing the symptoms. Vehicle computing system 302 may determine that patient 102 is experiencing the medical condition (“YES” branch of 706). Vehicle computing system 302 may retrieve an autonomous navigation mode from the memory of vehicle 112 (e.g., memory 312 of vehicle computing system 302) (708). Each autonomous navigation mode may contain instructions that, when executed by vehicle computing system 302, causes vehicle computing system 302 to control vehicle 112 to perform one or more functions independent of any input by patient 102. The one or more functions may include, but are not limited to, outputting an alert to bystanders (e.g., via warning lights, horn system of vehicle 112), navigating vehicle 112 to an isolated location, navigating vehicle 112 to a medical care location, and/or navigating vehicle 112 to a specialist medical care location.

Vehicle computing system 302 may select and retrieve an autonomous navigation mode from a plurality of stored autonomous navigation modes based on the type and severity of the medical condition. For example, vehicle computing system 302 selects a first autonomous navigation mode to navigate vehicle 112 to an isolated location in response to an occurrence of a medical condition at a first severity level and selects a second autonomous navigation mode to navigate vehicle 112 to a medical care facility in response to an occurrence of the medical condition at a higher severity level. In some examples, vehicle computing system 302 may determine a severity score for the medical condition based on the type and severity of the medical condition and may select an autonomous navigation mode corresponding to the severity score.

Vehicle computing system 302 causes vehicle control system 308 of vehicle 112 to autonomously navigate vehicle 112 in accordance with the retrieved autonomous navigation mode (710). Vehicle control system 308 may operate one or more systems of vehicle 112 (e.g., engine, steering system, braking system of vehicle 112) to move vehicle 112 along a route determined by vehicle computing system 302 from a current location of vehicle 112 to an intended location for vehicle 112. Vehicle computing system 302 may determine the route and navigate vehicle 112 along the route via location data retrieved from an external network (e.g., network 122) and/or via sensor data from one or more sensor(s) 304 disposed on an exterior of vehicle 112.

Vehicle computing system 302 may output notifications to one or more computing devices, systems, cloud computing environments, or networks (e.g., external device 120, network 122) to request medical aid to patient 102 and/or to prepare medical care providers for provision of the medical aid to patient 102.

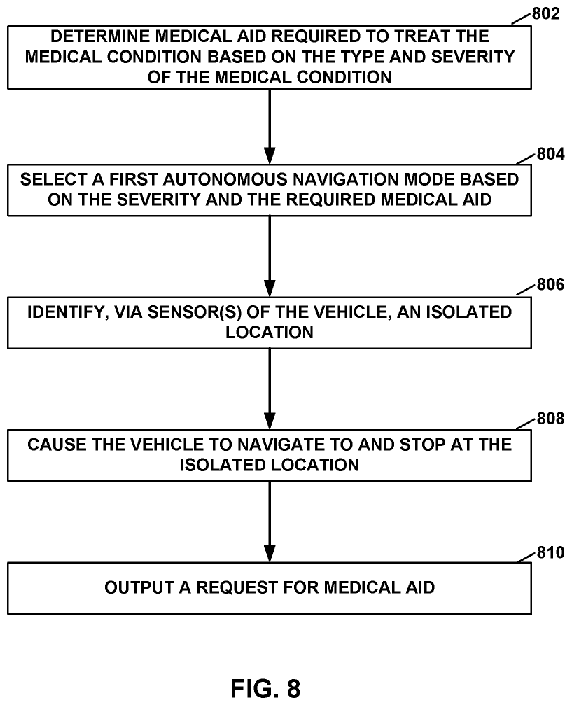

FIG. 8 is a flowchart illustrating another example process of autonomously navigating a vehicle 112 based on a determination that a patient 102 is experiencing a medical condition. Based on the type and severity of the medical condition, a clinician may deliver different types of medical aid to patient 102. In some examples, where patient 102 does not require life-critical medical care or immediate medical care, system 100 may reach out to nearby bystanders trained to deliver medical aid to render assistance to patient 102.

System 100 may determine a type of medical aid required to treat the medical condition based on the type and severity of the medical condition (802). Types of medical aid may include, but are not limited to, chest compressions, resuscitation, defibrillation of heart of patient 102, delivery of therapeutic substances (e.g., epinephrine to treat allergic reactions, naloxone to treat an opioid overdose, delivery of fluids, delivery of glucose to treat hypoglycemia), or delivery of oxygen to patient 102. Some types of medical aid may be appropriate for some types and severities of medical condition and may be inappropriate for other types and severities of medical condition. For example, if patient 102 is experiencing arrhythmia, the clinician does not deliver glucose to patient 102 to treat the arrhythmia. Similarly, if patient 102 is experiencing hypoglycemia, the clinician does not defibrillate the heart of patient 102.

System 100 may determine the type of medical aid required based on predetermined responses stored within system 100 (802). In some examples, vehicle 112 may transmit the type and the severity of the medical condition to a clinician (e.g., via external device 120) and/or to a medical care network (e.g., network 122). Vehicle 112 may then receive the appropriate type of medical aid for patient 102. In some examples, vehicle 112 retrieves the type of required medical aid from within memory of IMD 104 and/or of vehicle 112.

System 100 may select a first autonomous navigation mode based on the severity of the medical condition and the required medical aid (804). In some examples, the medical aid may be capable of being render by a nearby bystander and may be delivered in a shorter amount of time by a nearby bystander. In such examples, system 100 selects the first autonomous navigation mode to navigate vehicle 112 to an isolated location (e.g., out of the path of travel of other motorists, pedestrians, or other bystanders) to reduce a risk of collision between bystanders and vehicle 112. System 100 may then request bystanders to render medical aid to patient 102.

As a part of executing the first autonomous navigation mode, vehicle 112 may identify, via sensor(s) 304 on vehicle 112, an isolated location (806). The isolated location may include, but are not limited to, a driveway, a parking lot, side of a road, an empty street, a flat field next to a road, or any other location outside of the paths of travel of bystanders. The isolated location may be within visual and/or auditory range of passing bystanders (e.g., of passing motorists on a road). In some examples, vehicle 112 selects isolated locations that are within visual and/or auditory range of locations likely to contain people (e.g., next to hotels, hospitals, restaurants, businesses). In some examples, vehicle 112 retrieves, e.g., from an external network (e.g., network 122), location data (e.g., GPS data) corresponding to the current location of vehicle 112 and to isolated locations. Vehicle 112 (e.g., vehicle computing system 302) may determine a route from vehicle 112 to the isolated location. The route may be a quickest route or shortest route to the isolated location. Vehicle 112 may determine the route based on data from sensor(s) 304 and/or location data from the external network.

System 100 may cause vehicle to navigate to and to stop at the isolated location (808). Vehicle computing system 302 may cause vehicle control system 308 to move vehicle 112 along a determined route to the isolated location. Once vehicle computing system 302 determines that vehicle 112 is at the isolated location, vehicle computing system 302 to cause vehicle control system 308 to engage a brake system of vehicle 112 to park vehicle 112 at the isolated location.

Vehicle 112 may output a request for medical aid (810). In some examples, vehicle 112 transmits, via communications circuitry 118, a request for medical aid to external device 120 and/or to network 122. The request may include a location of vehicle 112, medical information on patient 102, the type and severity of the medical condition, and other information to facilitate a medical care provider to locate and treat patient 102. The request may be delivered to users capable of rendering medical aid to patient 102 including, but are not limited to, caretakers of patient 102, clinicians, EMS providers, users with medical training connected to network 122 and/or in connection with external device 120.

Vehicle 112 may transmit the request for medical aid to bystanders near vehicle 112. Vehicle 112 may transmit the request to computing devices, systems, or vehicles within a range of vehicle 112 (e.g., within a communications range of communications circuitry 118. In some examples, vehicle 112 alerts bystanders that patient 102 requires medical aid via visual and/or auditory indicators on vehicle 112 including, but are not limited to, warning lights or horns.

In some examples, IMD 104 may be configured to render the medical aid to patient 102. System 100 may cause IMD 104 to deliver the medical aid while vehicle 112 is parked at the isolated location. System 100 may determine whether the medical aid is effective and may select another autonomous navigation mode based on a determination that the medical aid is not effective. System 100 may relinquish control of vehicle 112 back to patient 102 based on a determination that the medical aid is effective and that patient 102 is capable of operating vehicle 112.

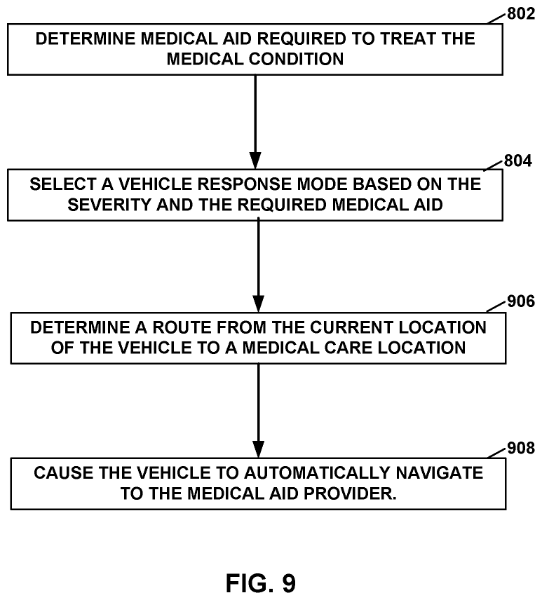

FIG. 9 is a flowchart illustrating another example process of autonomously navigating a vehicle 112 based on a determination that a patient 102 is experiencing a medical condition. In some examples, as opposed to the example illustrated in FIG. 8, patient 102 may require immediate medical aid and/or may require medical aid rendered by specific types of medical care providers and/or specific types of medical devices. In such examples, system 100 may cause vehicle 112 to navigate to a medical care facility in accordance with an autonomous navigation mode different from the example autonomous navigation mode described with regard to FIG. 8.

System 100 may determine medical aid required to treat the medical condition (804) and select an autonomous navigation mode based on the severity of the medical aid and the type of the medical aid (804). System 100 may determine the medical aid and select the navigation mode in accordance with the other example techniques previously described herein.

System 100 may determine a route from the current location of vehicle 112 to a medical care facility (906). Medical care facilities may include, but are not limited to, medical clinics, hospitals, EMS locations, locations with on-site medical devices (e.g., locations with AEDs), or specialist medical centers (e.g., medical centers specializing in treating certain type(s) of medical conditions such as strokes or cardiac conditions). System 100 may select the medical care facility from a plurality of medical care facilities based on a relative proximity of vehicle 112, based on a type of medical equipment available at the medical care location, and/or based on a specialization of the medical care location. System 100 may select the medical care facility based on the type and severity of the medical condition and the capability of each available medical care facility to render medical aid to treat the medical condition.

System 100 may cause vehicle 112 to automatically navigate to the medical care facility (908). System 100 may navigate vehicle 112 along a determine route from a current location of vehicle 112 to the medical care facility via sensor data from sensor(s) 304 on vehicle 112 and/or location data from one or more networks, e.g., as previously described herein.

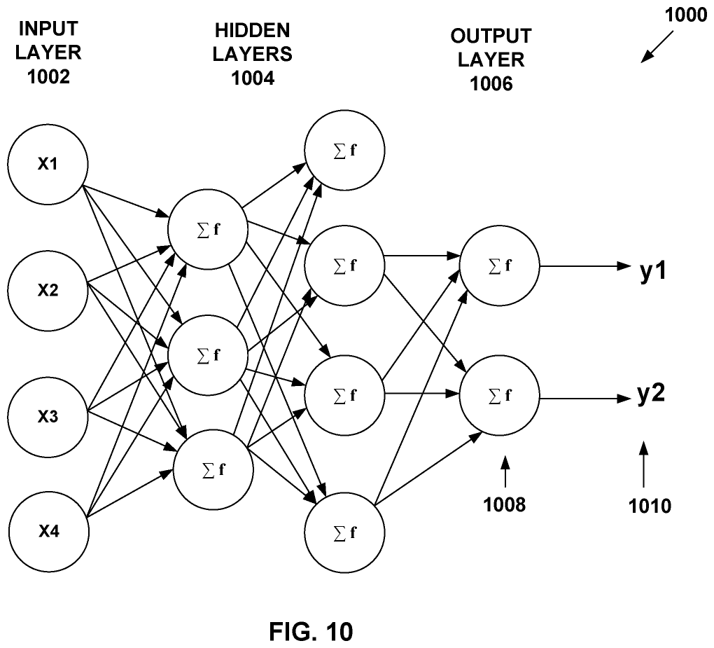

FIG. 10 is a conceptual diagram illustrating an example machine learning (ML) model 1000 configured to determine whether the patient 102 is experiencing a medical condition. While FIG. 10 primarily describes a ML model 1000 configured to determine whether patient 102 is experiencing a medical condition, another ML model may use a similar technique to determine a type of medical aid required by patient 102 and/or to select an autonomous navigation mode based on a type and/or a severity of a medical condition experienced by patient 102.

ML model 1000 is an example of a set of rules, e.g., a set of rules implemented by processing circuitry 106 of IMD 104 of system 100, as discussed above. ML model 1000 is an example of a deep learning model, or deep learning algorithm, trained to determine whether a particular set of physiological parameter data indicates the presence of a medical condition, e.g., whether a particular segment of ECG signal data indicates that patient 102 is experiencing arrhythmia. In some examples, ML model 1000 is trained to determine a severity level (e.g., an SOI score) of the medical condition One or more of IMD 104, vehicle 112 external device 120, or network 122 may train, store, and/or utilize ML model 1000, but other devices, systems, or cloud computing environments may apply inputs associated with a particular patient to ML model 1000 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 10, ML model 1000 may include three layers. These three layers include input layer 1002, hidden layer 1004, and output layer 1006. Output layer 1006 comprises the output from the transfer function 1008 of output layer 1006. Input layer 1002 represents each of the input values X1 through X4 provided to ML model 1000. The number of inputs may be less than or greater than 4, including much greater than 4, e.g., hundreds or thousands. In some examples, the input values may include any of the of values input into a machine learning model, as described above. In some examples, input values may include samples of an ECG signal, of a physiological signal, or physiological parameter values. In addition, in some examples input values of ML model 1000 may include additional data, such as data relating to one or more additional parameters (e.g., physiological characteristics) of patient 102.

Each of the input values for each node in the input layer 1002 is provided to each node of hidden layer 1004. In the example of FIG. 10, hidden layers 1004 include two layers, one layer having four nodes and the other layer having three nodes, but fewer or greater number of nodes may be used in other examples. Each input from input layer 1002 is multiplied by a weight and then summed at each node of hidden layers 1004. During training of ML model 1000, the weights for each input are adjusted to establish the relationship between the inputs, e.g., input ECG segment, to determining whether a particular set of inputs represents an acute health event and/or determining a score indicative of whether a set of inputs may be representative of SCA or another acute health event. In some examples, one hidden layer may be incorporated into ML model 1000, or three or more hidden layers may be incorporated into ML model 1000, where each layer includes the same or different number of nodes.

The result of each node within hidden layers 1004 is applied to the transfer function of output layer 1006. The transfer function may be liner or non-linear, depending on the number of layers within ML model 1000. Example non-linear transfer functions may be a sigmoid function or a rectifier function. The output 1010 of the transfer function may be a classification that indicates whether the particular ECG segment or other input set represents an acute health event and/or a score indicative of an extent to which the input data set represents an acute health event.

By applying the ECG signal data and/or other patient parameter data to a machine learning model, such as ML model 1000, processing circuitry, such as processing circuitry 106 of IMD 104, is able to determine a patient is experiencing or will soon experience an acute health event with great accuracy, specificity, and sensitivity. This may facilitate determinations of risk of sudden cardiac death, and may lead to alerts and other interventions as described herein.

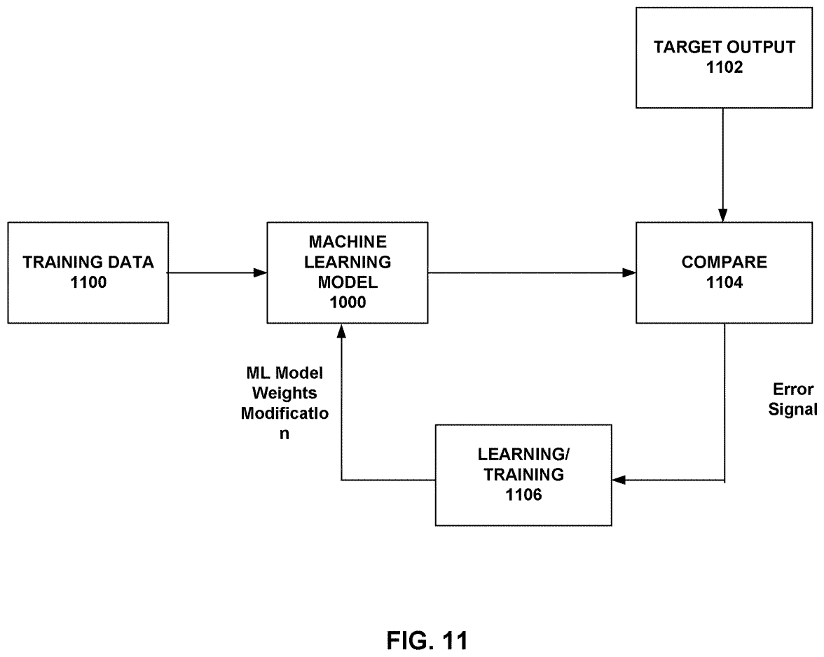

FIG. 11 is a conceptual diagram illustrating an example training process for an artificial intelligence model, in accordance with examples of the current disclosure. ML model 1000 may be implemented using any number of models for supervised and/or reinforcement learning, such as but not limited to, an artificial neural network, a decision tree, naïve Bayes network, support vector machine, or k-nearest neighbor model, to name only a few of the examples discussed above. In some examples, processing circuitry of one or more of IMD 104, vehicle 112, external device 120, network 122, and/or other computing devices, systems or cloud computing environments initially trains the ML model 1000 based on training set data 1100 including numerous instances of input data corresponding to acute health events and non-acute health events, e.g., as labeled by an expert. A prediction or classification by ML model 1000 may be compared 1104 to the target output 1102, e.g., as determined based on the label. Based on an error signal representing the comparison, the processing circuitry implementing a learning/training function 1106 may send or apply a modification to weights of ML model 1000 or otherwise modify/update the ML model 1000. For example, one or more of IMD 104, vehicle 112, external device 120, network 122, and/or other computing devices, systems or cloud computing environments may, for each training instance in the training set 1100, modify ML model 1000 to change an output generated by the ML model 1000 in response to data applied to the ML model 1000.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following is a non-limiting list of examples that are in accordance with one or more aspects of this disclosure.

Example 1: An implantable medical device (IMD) comprising: one or more sensors; sensing circuitry configured to sense, via the one or more sensors, a physiological signal of a patient; communications circuitry configured to wirelessly communicate with computing circuitry of a vehicle; and processing circuitry configured to: determine that the patient is operating the vehicle; adjust, based on a determination that the patient is operating the vehicle, a threshold condition for the sensed electrical signal; determine, whether the sensed electrical signal satisfies the adjusted threshold condition; and in response to a determination that the sensed electrical signal satisfies the adjusted threshold condition, cause the communications circuitry to transmit instructions to the computing circuitry to cause the vehicle to engage an autonomous-operating mode.

Example 2: the IMD of example 1, wherein one or more sensors comprises a plurality of electrodes operably connected to the patient.

Example 3: the IMD of any of examples 1 and 2, wherein the physiological signal of the patient comprises an electrocardiogram (ECG) signal of a heart of the patient.

Example 4: the IMD of example 3, wherein the threshold condition comprises a period of time where the sensing circuitry does not sense activity of the heart, and wherein to adjust the threshold condition for the sensed physiological signal, the processing circuitry is configured to reduce the period of time.

Example 5: the IMD of any of examples 3 and 4, wherein the threshold condition comprises a threshold detection criterion for tachyarrhythmia.

Example 6: the IMD of any of examples 3-5, wherein the threshold condition comprises a threshold amplitude of the ECG signal.

Example 7: the IMD of any of examples 1-6, wherein the processing circuitry is configured to determine, based on the determination that the sensed electrical signal satisfied the adjusted threshold condition, that the patient is experiencing a medical condition.

Example 8: the IMD of example 7, wherein the processing circuitry is configured to verify the determination that the patient is experiencing the medical condition by: transmitting, via the communications circuitry, request for sensor data from the computing circuitry of the vehicle; receiving, via the communications circuitry the sensor data from the computing circuitry; determining, based on the received sensor data, activity of the patient inside the vehicle; and determining whether the determined activity corresponds to symptoms of the medical condition.

Example 9: the IMD of any of examples 7 and 8, wherein the medical condition comprises arrhythmia.

Example 10: the IMD of any of examples 7-9, wherein the medical condition comprises a stroke.

Example 11: the IMD of any of examples 7-10, wherein the medical condition comprises hypoglycemia.

Example 12: the IMD of any of examples 1-11, wherein to determine that the patient is operating the vehicle, the processing circuitry is configured to determine that the communications circuitry is within communication range of the computing circuitry of the vehicle.

Example 13: the IMD of any of examples 1-12, wherein to adjust the threshold condition for the sensed electrical signal; the processing circuitry is configured to increase sensitivity of detection of the satisfaction of the threshold condition.

Example 14: the IMD of any of examples 1-13, wherein the processing circuitry is configured to apply a machine learning technique to adjust the threshold condition.

Example 15: the IMD of example 14, wherein the machine learning technique comprises a machine learning model configured to be trained via a training set comprising a plurality of sensed electrical signals of vehicle operators and a corresponding plurality of threshold conditions.

Example 16: the IMD of any of examples 1-15, wherein instructions to cause computing circuitry of the vehicle to engage in the autonomous-operating mode comprises instructions that, when executed by the computing circuitry, is configured to cause the vehicle to autonomously identify a location and navigate to the location.

Example 17: the IMD of example 16, wherein the location comprises a location of a medical care provider.

Example 18: the IMD of example 16, wherein the location comprises a parking location.

Example 19: the IMD of any of examples 1-18, wherein, in response to the determination that the sensed electrical signal satisfies the adjusted threshold condition, the processing circuitry is configured to cause the communications circuitry to transmit instructions to the computing circuitry to output a request for medical aid.

Example 20: a vehicle comprising: control circuitry configured to autonomously operate the vehicle; communications circuitry configured to wirelessly communicate with an implantable medical device (IMD); and computing circuitry configured to: determine, via one or more sensors in communication with the computing circuitry, that a patient is operating the vehicle; receive, from the IMD and via the communications circuitry, an indication that the patient is experiencing a medical condition based on a determination that a physiological signal sensed by the IMD satisfies a threshold condition; determine, based at least in part on the received indication, a severity of the medical condition; select, based on a determined severity of the medical condition, an autonomous-operating mode from a plurality of autonomous-operating modes; and cause the control circuitry to autonomously operate the vehicle based on the selected autonomous-operating mode.

Example 21: the vehicle of example 20, wherein the one or more sensors are disposed within a cabin of the vehicle, and wherein the one or more sensors are operably coupled to the computing circuitry.

Example 22: the vehicle of any of examples 20 and 21, wherein the one or more sensors comprise one or more of a camera, a microphone, or a pressure sensor.

Example 23: the vehicle of any of examples 20-22, wherein the computing circuitry is configured to: sense, via the one or more sensors, activity of the patient inside the vehicle; and determine whether the sensed activity of the patient corresponds to one or more symptoms of the medical condition.

Example 24: the vehicle of example 23, wherein the computing circuitry is configured to determine, based on a determination that the sensed activity of the patient does not correspond to the one or more symptoms of the medical condition, that the received indication is a false-positive.

Example 25: the vehicle of any of examples 23 and 24, wherein the computing circuitry is configured to determine, based on a magnitude of the sensed activity, and a determination that the sensed activity corresponds to at least one symptom of the medical condition, the severity of the medical condition.

Example 26: the vehicle of any of examples 20-25, wherein the physiological signal comprises an electrocardiogram (ECG) signal, and wherein the medical condition comprises a cardiac condition.

Example 27: the vehicle of example 26, wherein the cardiac condition comprises arrhythmia.

Example 28: the vehicle of any of examples 26 and 27, wherein to determine the severity of the medical condition, the computing circuitry is configured to determine whether the patient is currently experiencing cardiac arrest.

Example 29: the vehicle of any of examples 20-28, wherein the computing circuitry is configured to: based on a determination that the patient is currently experiencing the medical condition at a first level of severity, select a first autonomous-operating mode from the plurality of autonomous-operating modes, and based on a determination that the patient is currently experiencing the medical condition at a second level of severity, select a second autonomous-operating mode from the plurality of autonomous-operating modes, wherein the first autonomous-operating mode is different from the second autonomous-operating mode, and wherein the first level of severity comprises a different severity of illness (SOI) score than the second level of severity.

Example 30: the vehicle of example 29, wherein to autonomously operate the vehicle based on the selected first autonomous-operating mode, the control circuitry is configured to: autonomously cause the vehicle to stop; and output, via the communications circuitry, a request for medical aid.

Example 31: the vehicle of example 29, wherein the control circuitry is configured to output a request for medical aid to an emergency medical services (EMS) network.

Example 32: the vehicle of any of examples 30 and 31, wherein the control circuitry is configured to output the request for medical aid to one or more users in a network.

Example 33: the vehicle of example 29, wherein to autonomously operate the vehicle based on the selected first autonomous-operating mode, the computing circuitry is configured to: receive, from one or more networks and via the communications circuitry, a location of the vehicle and a location of an automated external defibrillator (AED) within a predetermined radius of the location of the vehicle; and cause the control circuitry to autonomously navigate the vehicle to the location of the AED.

Example 34: the vehicle of any of examples 29-33, wherein to autonomously operated the vehicle based on the selected second autonomous-operating mode, the computing circuitry is configured to: receive, from one or more networks and via the communications circuitry, a location of the vehicle and a location of a medical care facility closest to the location of the vehicle; and cause the control circuitry to autonomously navigate the vehicle to the location of the medical care facility.

Example 35: the vehicle of any of examples 29-33, wherein to autonomously operated the vehicle based on the selected second autonomous-operating mode, the computing circuitry is configured to: receive, from one or more networks and via the communications circuitry, a location of the vehicle, locations of a first medical care facility and a second medical care facility, wherein the first medical care facility is closer to the location of the vehicle than the second medical care facility, and wherein the second medical care facility specializes in treatment of the medical condition; and cause the control circuitry to autonomously navigate the vehicle to the location of the second medical care facility.

Example 36: a method for controlling operation of an implantable medical device (IMD) comprising: sensing, by sensing circuitry included in a housing of the IMD and via one or more sensors coupled to the sensing circuitry, a physiological signal of a patient, wherein the sensing circuitry and the one or more sensors are operably coupled to a processing circuitry included in the housing of the IMD; determining, by the processing circuitry, whether the patient is operating a vehicle; adjusting, by the processing circuitry and based on a determination that the patient is operating the vehicle, a threshold condition for the sensed electrical signal; determining, by the processing circuitry, whether the sensed electrical signal satisfies the adjusted threshold condition; and based on a determination that the sensed electrical signal satisfies the adjusted threshold condition, transmitting, by the processing circuitry and via communications circuitry operably coupled to the processing circuitry, instructions to cause computing circuitry of the vehicle to engage an autonomous-operating mode.

Example 37: the method of example 36, wherein the one or more sensors comprises a plurality of electrodes operably connected to the patient.

Example 38: the method of any of examples 36 and 37, wherein the physiological signal of the patient comprises an electrocardiogram (ECG) signal of a heart of the patient.

Example 39: the method of example 38, wherein the threshold condition comprises a period of time when the sensing circuitry does not sense activity of the heart, and wherein adjusting the threshold condition for the sensed physiological signal comprises reducing, by the processing circuitry, the period of time.

Example 40: the method of any of examples 38 and 39, wherein the threshold condition comprises a threshold detection criterion for tachyarrhythmia.

Example 41: the method of any of examples 38-40, wherein the threshold condition comprises a threshold amplitude of the ECG signal.

Example 42: the method of any of examples 36-41, further comprising determining, by the processing circuitry and based on the determination that the sensed electrical signal satisfied the adjusted threshold condition, that the patient is experiencing a medical condition.

Example 43: the method of example 42, further comprising verifying, by the processing circuitry, the determination that the patient is experiencing the medical condition, wherein verifying the determination comprises: transmitting, by the processing circuitry and via the communications circuitry, a request for sensor data from the computing circuitry of the vehicle; receiving, by the processing circuitry and via the communications circuitry, the sensor data from the computing circuitry; determining, by the processing circuitry and based on the received sensor data, activity of the patient inside the vehicle; and determining, by the processing circuitry, whether the determined activity corresponds to symptoms of the medical condition.

Example 44: the method of any of examples 42 and 43, wherein the medical condition comprises arrhythmia.

Example 45: the method of any of examples 36-44, wherein determining whether the patient is operating the vehicle comprises: determining, by the processing circuitry, whether the communications circuitry is within communication range of the computing circuitry of the vehicle.

Example 46: the method of any of examples 36-45, wherein adjusting the threshold condition for the sensed electrical signal comprises increasing, by the processing circuitry, sensitivity of detection of satisfaction of the threshold condition.

Example 47: the method of any of examples 36-46, wherein adjusting the threshold condition comprises applying, by the processing circuitry, a machine learning technique to adjust the threshold condition.

Example 48: the method of example 47, wherein the machine learning technique comprises a machine learning model configured to be trained via a training set comprising a plurality of sensed electrical signals of vehicle operators and a corresponding plurality of threshold conditions.

Example 49: the method of any of examples 36-47, wherein the instructions to cause computing circuitry of the vehicle to engage in the autonomous-operating mode comprises instructions that, when executed by the computing circuitry, causes the vehicle to autonomously identify a location and navigate to the location.

Example 50: the method of example 49, wherein the location comprises a location of a medical care provider.

Example 51: the method of example 49, wherein the location comprises a parking location.

Example 52: a method for controlling operation of computing circuitry of a vehicle comprising: determining, by the computing circuitry and via one or more sensors in communication with the computing circuitry, that a patient is currently operating the vehicle; receiving, by the computing circuitry and via communications circuitry of the vehicle, an indication that a patient is experiencing a medical condition based on a determination that a physiological signal sensed by an implantable medical device (IMD) coupled to the patient satisfies a threshold condition; determining, by the computing circuitry and based at least in part on the received indication, a severity of the medical condition; selecting, by the computing circuitry and based on a determined severity of the medical condition, an autonomous-operating mode from a plurality of autonomous-operating modes; and causing, by the computing circuitry, control circuitry of the vehicle to autonomously operate the vehicle based on the selected autonomous-operating mode.

Example 53: the method of example 52, wherein the one or more sensors within a cabin of the vehicle, wherein the one or more sensors are operably coupled to the computing circuitry, and wherein the method further comprises: sensing, by the computing circuitry and via the one or more sensors, activity of the vehicle inside the vehicle; and determining, by the computing circuitry, whether the sensed activity of the patient corresponds to one or more symptoms of the medical condition.

Example 54: the method of example 53, wherein the one or more sensors comprise one or more of a camera, a microphone, or a pressure sensor.

Example 55: the method of any of examples 53 and 54, further comprising: determining, by the computing circuitry and based on a determination that the sensed activity of the patient does not correspond to the one or more symptoms of the medical condition, that the received indication is a false-positive.

Example 56: the method of any of examples 53-55, further comprising: determining, by the computing circuitry and based on a magnitude of the sensed activity and a determination that the sensed activity corresponds to at least one symptom of the medical condition, the severity of the medical condition.

Example 57: the method of any of examples 52-56, wherein the physiological signal comprises an electrocardiogram (ECG) signal, and wherein the medical condition comprises a cardiac condition.

Example 58: the method of example 57, wherein the cardiac condition comprises arrhythmia.

Example 59: the method of any of examples 57 and 58, wherein determining the severity of the medical condition comprising determining, by the computing circuitry, whether the patient is currently experiencing cardiac arrest.

Example 60: the method of any of examples 52-59, wherein selecting, the autonomous-operating mode from the plurality of autonomous-operating modes comprises: selecting, by the computing circuitry and based on a determination that the patient is experiencing the medical condition at a first level of severity, a first autonomous-operating mode from the plurality of autonomous-operating modes; and selecting, by the computing circuitry and based on a determination that the patient is experiencing the medical condition at a second level of severity, a second autonomous-operating mode from the plurality of autonomous-operating modes, wherein the first autonomous-operating mode is different from the second autonomous-operating mode, and wherein the first level of severity comprises a different severity of illness (SOI) score than the second level of severity.

Example 61: the method of example 60, wherein autonomously operating the vehicle based on the selected first autonomous-operating mode comprises: autonomously causing, by the control circuitry, the vehicle to stop; and outputting, by the control circuitry and via the communications circuitry, a request for medical aid.

Example 62: the method of example 61, wherein outputting the request for medical aid comprises outputting, by the control circuitry, the request to an emergency medical services (EMS) network.

Example 63: the method of any of examples 61 and 62, wherein outputting the request for medical aid comprises outputting, by the control circuitry, the request for medical aid to one or more users in a network.

Example 64: the method of example 60, wherein autonomously operating the vehicle based on the selected first autonomous-operating mode comprises: receiving, by the computing circuitry and from one or more networks, a location of the vehicle and a location of an automated external defibrillator (AED) within a predetermined radius of the location of the vehicle; and causing, by the computing circuitry, the control circuitry to autonomously navigate the vehicle to the location of the AED.

Example 65: the method of any of examples 60-64, wherein autonomously operating the vehicle based on the selected second autonomous-operating mode comprises: receiving, by the computing circuitry and from one or more networks, a location of the vehicle and a location of a medical care facility closest to the location of the vehicle; and causing, by the computing circuitry, the control circuitry to autonomously navigate the vehicle to the location of the medical care facility.

Example 66: the method of any of examples 60-64, wherein autonomously operating the vehicle based on the selected second autonomous-operating mode comprises: receiving, by the computing circuitry and from one or more networks, a location of the vehicle, locations of a first medical care facility and a second medical care facility, wherein the first medical care facility is closer to the location of the vehicle than the second medical care facility, and wherein the second medical care facility specializes in treatment of the medical condition; and causing, by the computing circuitry, the control circuitry to autonomously navigate the vehicle to the location of the second medical care facility.

Example 67: a computer-readable medium that, when executed by processing circuitry of an implantable medical device (IMD), is configured to cause the processing circuitry to perform the method of any of examples 36-51.

Example 68: a computer-readable medium that, when executed by processing circuitry of an implantable medical device (IMD), is configured to cause the processing circuitry to perform the method of any of examples 52-66.

Example 69: a system comprising: an implantable medical device (IMD) of any of examples 1-19; and a vehicle of any of examples 20-35.

1: An implantable medical device (IMD) comprising:
one or more sensors;
sensing circuitry configured to sense, via the one or more sensors, a physiological signal of a patient;
communications circuitry configured to wirelessly communicate with computing circuitry of a vehicle; and
processing circuitry configured to:
determine that the patient is operating the vehicle;
adjust, based on a determination that the patient is operating the vehicle, a threshold condition for the sensed electrical signal;
determine, whether the sensed electrical signal satisfies the adjusted threshold condition; and
in response to a determination that the sensed electrical signal satisfies the adjusted threshold condition, cause the communications circuitry to transmit instructions to the computing circuitry to cause the vehicle to engage an autonomous-operating mode.

2: The IMD of claim 1, wherein the physiological signal of the patient comprises an electrocardiogram (ECG) signal of a heart of the patient.

3: The IMD of claim 2, wherein the threshold condition comprises a period of time where the sensing circuitry does not sense activity of the heart, and wherein to adjust the threshold condition for the sensed physiological signal, the processing circuitry is configured to reduce the period of time.

4: The IMD of claim 1, wherein the processing circuitry is configured to determine, based on the determination that the sensed electrical signal satisfied the adjusted threshold condition, that the patient is experiencing a medical condition.

5: The IMD of claim 4, wherein the processing circuitry is configured to verify the determination that the patient is experiencing the medical condition by:
transmitting, via the communications circuitry, request for sensor data from the computing circuitry of the vehicle;
receiving, via the communications circuitry the sensor data from the computing circuitry;
determining, based on the received sensor data, activity of the patient inside the vehicle; and
determining whether the determined activity corresponds to symptoms of the medical condition.

6: The IMD of claim 1, wherein instructions to cause computing circuitry of the vehicle to engage in the autonomous-operating mode comprises instructions that, when executed by the computing circuitry, is configured to cause the vehicle to autonomously identify a location and navigate to the location.

7: A vehicle comprising:
control circuitry configured to autonomously operate the vehicle;
communications circuitry configured to wirelessly communicate with an implantable medical device (IMD); and
computing circuitry configured to:
determine, via one or more sensors in communication with the computing circuitry, that a patient is operating the vehicle;
receive, from the IMD and via the communications circuitry, an indication that the patient is experiencing a medical condition based on a determination that a physiological signal sensed by the IMD satisfies a threshold condition;
determine, based at least in part on the received indication, a severity of the medical condition;
select, based on a determined severity of the medical condition, an autonomous-operating mode from a plurality of autonomous-operating modes; and
cause the control circuitry to autonomously operate the vehicle based on the selected autonomous-operating mode.

8: The vehicle of claim 7, wherein the computing circuitry is configured to:
sense, via the one or more sensors, activity of the patient inside the vehicle; and
determine whether the sensed activity of the patient corresponds to one or more symptoms of the medical condition.

9: The vehicle of claim 8, wherein the computing circuitry is configured to determine, based on a determination that the sensed activity of the patient does not correspond to the one or more symptoms of the medical condition, that the received indication is a false-positive.

10: The vehicle of claim 8, wherein the computing circuitry is configured to determine, based on a magnitude of the sensed activity, and a determination that the sensed activity corresponds to at least one symptom of the medical condition, the severity of the medical condition.

11: The vehicle of claim 7, wherein the computing circuitry is configured to:
based on a determination that the patient is currently experiencing the medical condition at a first level of severity, select a first autonomous-operating mode from the plurality of autonomous-operating modes, and
based on a determination that the patient is currently experiencing the medical condition at a second level of severity, select a second autonomous-operating mode from the plurality of autonomous-operating modes,
wherein the first autonomous-operating mode is different from the second autonomous-operating mode, and wherein the first level of severity comprises a different severity of illness (SOI) score than the second level of severity.

12: The vehicle of claim 11, wherein to autonomously operate the vehicle based on the selected first autonomous-operating mode, the control circuitry is configured to:
autonomously cause the vehicle to stop; and
output, via the communications circuitry, a request for medical aid.

13: The vehicle of claim 11, wherein to autonomously operate the vehicle based on the selected first autonomous-operating mode, the computing circuitry is configured to:
receive, from one or more networks and via the communications circuitry, a location of the vehicle and a location of an automated external defibrillator (AED) within a predetermined radius of the location of the vehicle; and
cause the control circuitry to autonomously navigate the vehicle to the location of the AED.

14: The vehicle of claim 11, wherein to autonomously operated the vehicle based on the selected second autonomous-operating mode, the computing circuitry is configured to:
receive, from one or more networks and via the communications circuitry, a location of the vehicle and a location of a medical care facility closest to the location of the vehicle; and
cause the control circuitry to autonomously navigate the vehicle to the location of the medical care facility.

15: The vehicle of claim 11, wherein to autonomously operated the vehicle based on the selected second autonomous-operating mode, the computing circuitry is configured to:
receive, from one or more networks and via the communications circuitry, a location of the vehicle, locations of a first medical care facility and a second medical care facility, wherein the first medical care facility is closer to the location of the vehicle than the second medical care facility, and wherein the second medical care facility specializes in treatment of the medical condition; and
cause the control circuitry to autonomously navigate the vehicle to the location of the second medical care facility.

16: A method for controlling operation of an implantable medical device (IMD) comprising:
sensing, by sensing circuitry included in a housing of the IMD and via one or more sensors coupled to the sensing circuitry, a physiological signal of a patient, wherein the sensing circuitry and the one or more sensors are operably coupled to a processing circuitry included in the housing of the IMD;
determining, by the processing circuitry, whether the patient is operating a vehicle;
adjusting, by the processing circuitry and based on a determination that the patient is operating the vehicle, a threshold condition for the sensed electrical signal;
determining, by the processing circuitry, whether the sensed electrical signal satisfies the adjusted threshold condition; and
based on a determination that the sensed electrical signal satisfies the adjusted threshold condition, transmitting, by the processing circuitry and via communications circuitry operably coupled to the processing circuitry, instructions to cause computing circuitry of the vehicle to engage an autonomous-operating mode.

17: The method of claim 16, wherein the physiological signal of the patient comprises an electrocardiogram (ECG) signal of a heart of the patient, and wherein the threshold condition comprises a period of time when the sensing circuitry does not sense activity of the heart, and wherein adjusting the threshold condition for the sensed physiological signal comprises reducing, by the processing circuitry, the period of time.

18: The method of claim 16, further comprising:
determining, by the processing circuitry and based on the determination that the sensed electrical signal satisfied the adjusted threshold condition, that the patient is experiencing a medical condition.

19: The method of claim 18, further comprising verifying, by the processing circuitry, the determination that the patient is experiencing the medical condition, wherein verifying the determination comprises:
transmitting, by the processing circuitry and via the communications circuitry, a request for sensor data from the computing circuitry of the vehicle;
receiving, by the processing circuitry and via the communications circuitry, the sensor data from the computing circuitry;
determining, by the processing circuitry and based on the received sensor data, activity of the patient inside the vehicle; and
determining, by the processing circuitry, whether the determined activity corresponds to symptoms of the medical condition.

20: The method of claim 16, wherein the instructions to cause computing circuitry of the vehicle to engage in the autonomous-operating mode comprises instructions that, when executed by the computing circuitry, causes the vehicle to autonomously identify a location and navigate to the location.
